SEQUENCE LISTING (9 sequences)

SEQ ID NO: 1 - DNA, 682 bp, Saccharomyces cerevisiae
ggatccggggttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgaaaattcgaat

SEQ ID NO: 2 - DNA, 180 bp, Saccharomyces cerevisiae
tattagtagctaaaaatgggtcacgtgatctatattcgaaaggggcggttgcctcaggaaggcaccggcggtctttcgtccgtgcggagatatctgcgccgttcaggggtccatgtgccttggacgatattaaggcagaaggcagtatcggggcggatcactccgaaccgagattaggta

SEQ ID NO: 3 - DNA, 392 bp, Saccharomyces cerevisiae
ggatccaagcttgcatgcctgcaggtcggagtactgtcctccgagcggagtactgtcctccgagcggagtactgtcctccgagcggagtactgtcctccgagcggagtactgtcctccgagcggagactctagcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagggaattggg

SEQ ID NO: 4 - DNA, 625 bp, Saccharomyces cerevisiae
ggagttcagtgataaaagtgtcacagcgaatttcctcatatgtagggaccgaattgtttacaagttctctgtaccaccatggagacatcaaagattgaaaatctatggaaagatatggacggtagcaacaagaatatagcacgagccgcggatttatttcgttacttttgatatcactcacaactattgcgaagcgcttcagtgaaaaaatcataaggaaaagttgtaaatattattggtactattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggagagggtccaaaaagcgctcggacaactgttgaccgtgatccgaaggactggctatacagtgttcacaaaatagccaagctgaaaataatgtgtagcctttagctatgttcagttagtttggctagcaaagatataaaagcaggtcggaaatatttatgggcattattatgcagagcatcaacatgataaaaaaaacagttgaatattccctcaaaaatg

SEQ ID NO: 5 - DNA, 232 bp, Saccharomyces cerevisiae
ggccatatgtcttccgaaagaagaagaaaggaagacatgtattacattatccaacaaaaaatggttcttgacgtctacaaatcaagaatcttaaagacattgaacgaagtagctgaataaaaattatgaaaactataaaaactataaaaactgtacttaagtcctcaataaaacataaacttcttactgtataaggttttcgataatttcttacttgattctaggagagcaa

SEQ ID NO: 6 - DNA, 541 bp, Saccharomyces cerevisiae
atccgaaagttgtattcaacaagaatgcgcaaatatgtcaacgtatttggaagtcatcttatgtgcgctgctttaatgttttctcatgtaagcggacgtcgtctataaacttcaaacgaaggtaaaaggttcatagcgctttttctttgtctgcacaaagaaatatatattaaattagcacgttttcgcatagaacgcaactgcacaatgccaaaaaaagtaaaagtgattaaaagagttaattgaataggcaatctctaaatgaatcgatacaaccttggcactcacacgtgggactagcacagactaaatttatgattctggtccctgttttcgaagagatcgcacatgccaaattatcaaattggtcaccttacttggcaaggcatatacccatttgggataagggtaaacatctttgaattgtcgaaatgaaacgtatataagcgctgatgttttgctaagtcgaggttagtatggcttcatctctcatgagaataagaacaacaacaaatagagcaagcaaattcgagattaccaa

SEQ ID NO: 7 - DNA, 17 bp, Artificial Sequence
  oligonucleotide
cggaggactgtcctccg

SEQ ID NO: 8 - DNA, 38 bp, Artificial Sequence
  oligonucleotide
atctgtacataaacccagtggttttatgtacagtatta

SEQ ID NO: 9 - DNA, 72 bp, Artificial Sequence
  cI operator sequence
tcgagcttgacattgtaggactatattgctctaataaatttgcggccgctaatacgactcactatagggaga

(19) United States
(12) Patent Application Publication
(10) Pub. No.: US 2009/0011405 A1
(43) Pub. Date: Jan. 8, 2009

(54) Modulating Screening Thresholds for N-Hybrid Screening

(76) Inventors: Paul Watt, Western Australia (AU); Richard Hopkins, Western Australia (AU); Vanessa Cull, Western Australia (AU); Mark Fear, Western Australia (AU); Nadia Milech, Western Australia (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/546,122

(22) PCT Filed: Feb. 20, 2004
(86) PCT No.: PCT/AU04/00216

§ 371 Date: Dec. 21, 2006

(30) Foreign Application Priority Data
Feb. 21, 2003 (AU) 2003900786

(51) Int. Cl. C12Q 1/68; G01N 33/53; G01N 33/569

(52) U.S. Cl. 435/6; 435/7.2; 435/7.31

(57) ABSTRACT

The present invention provides improved N-hybrid assays. In particular, the present invention provides an improved reverse N-hybrid assay comprising modulating the amount of a substrate of a reporter gene and/or the amount of a reporter gene thereby enhancing cell death in the absence of a peptide inhibitor of a DNA-protein or protein-protein interaction and/or enhancing cell survival in the presence of a peptide inhibitor of a DNA-protein or protein-protein interaction. Furthermore, the present invention provides an improved forward N-hybrid assay comprising modulating the amount of a reporter gene thereby enhancing cell death in the absence of a heterologous peptide or protein capable of binding to the DNA or protein in a cell and enhancing cell survival in the presence of a heterologous peptide or protein capable of binding to the DNA or protein in a cell.

### FIELD OF THE INVENTION

The present invention relates to improved N-hybrid assays. In particular, the present invention relates to an improved reverse N-hybrid assay comprising modulating the amount of a substrate of a reporter gene and/or the amount of a reporter gene thereby enhancing cell death in the absence of a peptide inhibitor of a DNA-protein or protein-protein interaction and/or enhancing cell survival in the presence of a peptide inhibitor of a DNA-protein or protein-protein interaction. Furthermore, the present invention relates to an improved forward N-hybrid assay comprising modulating the amount of reporter gene expression thereby enhancing cell death or inhibiting cell growth in the absence of a heterologous peptide or protein capable of binding to the DNA or protein in a cell and enhancing cell survival in the presence of a heterologous peptide or protein capable of binding to the DNA or protein in a cell.

### BACKGROUND OF THE INVENTION

### General

This specification contains nucleotide and amino acid sequence information prepared using PatentIn Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
Sambrook, J. and Russell, D. W., Molecular Cloning: A Laboratory Manual, Cold Spring Harbour Laboratory Press, Cold Spring Harbour, N.Y. Third Edition (2001), whole of Vols I, II, and III;
Ausubel, F. M., Brent, R, Kingston, R. E., Moore, D. D., Seidman, J. G., and Struhl, K. (Editors). Current Protocols in Molecular Biology, John Wiley and Sons, New York (1987-), whole of volumes DNA Cloning: A Practical Approach, Vols. I and II (D. N. Glover, ed., 1985), IRL Press, Oxford, whole of text;
Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
Nucleic Acid Hybridization: A Practical Approach (B. D. Hames & S. J. Higgins, eds., 1985) IRL Press, Oxford, whole of text;
Animal Cell Culture: Practical Approach, Third Edition (John R. W. Masters, ed., 2000), ISBN 0199637970, whole of text;
Immobilized Cells and Enzymes: A Practical Approach (1986) IRL Press, Oxford, whole of text;
Perbal, B., A Practical Guide to Molecular Cloning (1984);
Methods In Enzymology (S. Colowick and N. Kaplan, eds., Academic Press, Inc.), whole of series;
J. F. Ramalho Ortigão, “The Chemistry of Peptide Synthesis” In: Knowledge database of Access to Virtual Laboratory website (Interactiva, Germany);
Sakakibara, D., Teichman, J., Lien, E. Land Fenichel, R. L. (1976). Biochem. Biophys. Res. Commun. 73 336-342
Merrifield, R. B. (1963). J. Am. Chem. Soc. 85, 2149-2154.
Barany, G. and Merrifield, R. B. (1979) in The Peptides (Gross, E. and Meienhofer, J. eds.), vol. 2, pp. 1-284, Academic Press, New York.
Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
Bodanszky, M. (1984) Principles of Peptide Synthesis, Springer-Verlag, Heidelberg.
Bodanszky, M. & Bodanszky, A. (1984) The Practice of Peptide Synthesis, Springer-Verlag, Heidelberg.
Bodanszky, M. (1985) Int. J. Peptide Protein Res. 25, 449-474.
Handbook of Experimental Immunology, Vols. I-IV (1). M. Weir and C. C. Blackwell, eds., 1986, Blackwell Scientific Publications).

### DESCRIPTION OF THE RELATED ART

Biological interactions, such as, protein:protein interactions, protein:nucleic acid interactions and protein:ligand interactions are involved in awide variety of cellular processes. In fact, at least one of these interactions are critical to most biological processes, from formation of cellular macromolecular structures and enzymatic complexes, to the regulation of signal transduction pathways.

Traditionally such interactions were identified and characterised using time and labour intensive biochemical approaches, such as, for example, molecular cloning of genes encoding interacting proteins using expression library cloning.

The development of the N-hybrid assays provided the means for rapid screening to not only identify peptides, polypeptides and/or proteins involved in protein:protein interactions, protein:nucleic acid interactions and protein:ligand interactions (ie forward N-hybrid assays) but also inhibitors of these interactions (ie reverse N-hybrid assays). Such assays are based on the use of complementation to select for those cells that comprise a nucleic acid:protein interaction or a protein:protein interaction (in the case of a forward N-hybrid assay) or an inhibitor thereof (in the case of a reverse N-hybrid assay).

For example, a forward two-hybrid assay is used to determine a peptide, polypeptide or protein that is capable of interacting with a protein of interest. The assay is performed in a yeast cell that is auxotrophic, for example, the cell is unable to grow in the absence of leucine. These yeast cells also comprise a gene that confers the ability to grow in the absence of leucine under control of an inducible promoter. The protein of interest is expressed in a yeast cell as a fusion protein with a protein domain capable of binding to the inducible promoter. Another protein, or a library of proteins are then expressed as a fusion protein with a protein domain that is a transcriptional activation domain. Upon interaction of the protein of interest and another protein the reporter gene is activated, thereby conferring the ability to grow in the absence of leucine. Accordingly, only cells expressing a protein capable of interacting with the protein of interest grow in the absence of leucine.

A disadvantage of this system is that there are several proteins endogenous to a cell that may be capable of binding to a protein of interest and inducing activation of the reporter gene. This has lead to the identification of a large number of “false positives”.

To overcome this disadvantage, many researchers then perform the two hybrid assay in the opposite direction, ie an identified protein is expressed as a fusion with a DNA binding domain and the protein of interest is expressed as a fusion with a transcriptional activation domain. However, while this may reduce the number of false positives to a degree, the process is both time consuming and expensive.

This same limitation applies to methods of testing each identified interaction in vitro or in vivo using, for example, immunoprecipitation experiments.

In the case of a reverse two hybrid assay, a similar assay as the forward assay is used, however the reporter gene is a counter selectable marker, eg URA3. In the presence of 5-fluororotic acid (5-FOA), any expression of the URA3 reporter gene leads to production of a toxic compound. Accordingly, cells in which a protein:protein interaction occurs that induces expression of URA3 are killed in the presence of 5-FOA. Those cells that express an inhibitor of the protein:protein interaction do not express the URA3 gene product, and, as a consequence, survive in the presence of s-FOA.

Without detracting form the general applicability of the present invention, a major difficulty encountered in the generation of a reverse N-hybrid system arises from the ability of a significant proportion of proteins endogenously expressed in a cell to interact with one or other of the interacting proteins and activate expression of a counter-selectable marker.

A method that has been recently suggested to overcome this problem by reducing the sensitivity of the reporter molecule. This is achieved by reducing the number of activating sequences associated with a counter-selectable marker (as reviewed by White, Proc. Natl. Acad. Sci. USA, 93, 10001-10003, 1996), whilst maintaining sufficient sensitivity in the system to detect an antagonist of the protein-protein interaction. A disadvantage of this approach is that it is necessary to determine the number of activating sequences associated with a given selectable marker for each screen involving a different protein-protein or nucleic acid:protein interaction interaction.

Accordingly, there remains a need for methods that provide a rapid and inexpensive means for reducing background in both forward and reverse N-hybrid assays. Preferably, such a system does not significantly reduce the sensitivity of an assay, ie the screen maintains the plating efficiency of a N-hybrid assay. Such methods would be of particular use in developing more sensitive reporter systems for the analysis of protein functions, especially in the rapidly expanding field of identifying antagonists of protein interactions.

### SUMMARY OF INVENTION

In work leading up to the present invention the inventors sought to determine a method for reducing the background of a N-hybrid screen while maintaining or increasing the plating efficiency of the screen. Such a method not only reduces the number of false positives identified but also enables the screening of a larger number of cells in a single screen thereby facilitating the identification of more positive clones.

In particular the present inventors have found that by modulating the expression of a reporter gene by virtue of modulating the expression of one or more of the interacting partners that activate the expression of the reporter gene, the number of background colonies is reduced. Furthermore, the present inventors have shown that by modulating the substrate of a reporter gene, the background levels are reduced and the plating efficiency maintained or enhanced.

Accordingly, one aspect of the present invention provides an improved reverse N-hybrid assay for identifying a peptide inhibitor of a DNA-protein or protein-protein interaction comprising:
(a) determining an amount of a substrate or modulator of a reporter gene required to enhance cell death in the absence of a peptide inhibitor of the DNA-protein or protein-protein interaction; and/or
(b) determining an amount of a substrate or modulator of a reporter gene required to enhance cell survival in the presence of a peptide inhibitor of the DNA-protein or protein-protein interaction; and
(c) performing a reverse N-hybrid assay using an amount of the substrate or modulator as determined at (a) and (b),
wherein expression of the reporter gene modulates cell survival by virtue of encoding a polypeptide that reduces cell growth or viability by providing a target for a cytostatic or cytotoxic compound or by converting the substrate to a cytostatic or cytotoxic product.

As used herein, the term “a peptide inhibitor of a DNA-protein interaction or a protein-protein interaction” shall be taken to mean a peptide that is capable of binding to one or more binding partners on a DNA protein interaction or a protein-protein interaction and thereby disrupt said interaction. Preferably, the peptide inhibitor of a DNA-protein or protein-protein interaction is a heterologous peptide that is not endogenous to the cell or capable of modulating expression of the reporter gene.

In a preferred embodiment, the improved reverse N-hybrid assay is performed in a yeast cell.

In the context of a reverse N-hybrid the term “background number of cells” shall be taken to mean the number of cells that express the interacting protein/s of a protein-DNA interaction or the proteins of a protein-protein interaction and are capable of surviving in the absence of a peptide inhibitor of a DNA-protein or protein-protein interaction and in the presence of the substrate of the reporter gene.

Preferably, the background number of cells is determined using a method comprising
(a) culturing a known number of cells that expresses at least one interacting protein of a protein-DNA interaction or at least two proteins of a protein-protein interaction in the presence of a substrate of a reporter gene under conditions sufficient to produce cell death by virtue of reporter gene expression; and
(b) determining the number of cells that survive and/or grow wherein any cells that survive and/or grow in the presence of the substrate of the reporter gene are background.

In the context of a reverse N-hybrid assay the term “plating efficiency” shall be taken to mean the number of cells that express the interacting protein/s of a protein-DNA interaction or the proteins of a protein-protein interaction and are capable of surviving in the presence of a peptide inhibitor of a DNA-protein or protein-protein interaction and the substrate of the reporter gene.

Preferably, the plating efficiency is determined by a method comprising:
(a) culturing a known number of cells that express at least one interacting protein of a protein-DNA interaction and a peptide inhibitor of the protein-DNA interaction or at least two proteins of a protein-protein interaction and a peptide inhibitor of the protein interaction in the presence of a substrate of a reporter gene under conditions sufficient to produce cell death by virtue of reporter gene expression; and
(b) determining the number of cells that survive and/or grow compared to the number of cells cultured wherein a decrease in the number of cells that survive and/or grow compared to the number of cells cultured indicates a reduction in plating efficiency.

In one preferred embodiment, conditions sufficient to produce cell death by virtue of reporter gene expression comprise incubating the cells in an amount of a non-toxic (ie. toxigenic) substrate of the reporter gene under conditions sufficient for the reporter gene to be capable of being expressed in the cell.

By “toxigenic” is meant that the substrate can be converted to a toxic compound during performance of the assay to provide a selection.

In one embodiment, the cell is a yeast cell and the toxigenic substrate is 5-fluororotic acid (5-FOA) and the reporter gene is URA3.

In another embodiment, the cell is a yeast cell and the toxigenic substrate is cycloheximide and the reporter gene is CYH2.

In a further embodiment, the cell is a yeast cell and the toxigenic substrate is α-aminoadipate and the reporter gene is LYS2.

In a particularly preferred embodiment, conditions sufficient to produce cell death by virtue of reporter gene expression comprise incubating the cells in an amount of a plurality of toxigenic substrates of a plurality of reporter genes under conditions sufficient for each reporter gene to be capable of being expressed in the cell.

In one embodiment, the cell is a yeast cell and the plurality of reporter genes is selected from the group consisting of URA3, CYH2, LYS2 and wherein the plurality of toxigenic substrates is selected from the group consisting of 5-fluororotic acid, cycloheximide and α-aminoadipate.

In yet another embodiment conditions sufficient to produce cell death by virtue of reporter gene expression comprise incubating the cells in media lacking an amount of a compound required for cell survival and complemented by a product of reporter gene expression under conditions sufficient for the reporter gene to be capable of being expressed in the cell.

In a particularly preferred embodiment, the cell is a yeast cell and the compound required for cell survival is uracil and the reporter gene is URA3.

In a further embodiment, conditions sufficient to produce cell death by virtue of reporter gene expression comprise incubating the cells in an amount of a compound sufficient to modulate expression of a gene selected from the group consisting of a reporter gene and a gene encoding a binding partner to the DNA-protein or protein-protein interaction.

In a preferred embodiment, the gene encoding a binding partner comprises a promoter that is regulated by the compound thereby modulating expression of the reporter gene by virtue of modulating the amount of a binding partner that regulates reporter gene expression in the cell.

In another embodiment, the reporter gene comprises a promoter that is regulated by the compound thereby modulating expression of the reporter gene in the cell.

In a preferred embodiment, the gene encoding a binding partner or the reporter gene comprises a promoter that is induced in the presence of the compound.

In a particularly preferred embodiment the compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

The nucleotide sequences of the promoters referred to herein are available from the corresponding gene sequences as follows: the GAL1/GAL10 gene sequence is disclosed in NCBI database Accession No. K02115; the GAL2 gene sequence is disclosed in NCBI database Accession No. M81879; the GAL7 gene sequence is disclosed in NCBI database Accession No. X002151; and the MEL1 gene sequence is disclosed in NCBI database Accession No. X03102. Similarly, the GAL4 promoter is contained within a vector, the sequence of which is disclosed in NCBI database Accession No. AF140802.

In another preferred embodiment, the gene encoding a binding partner or the reporter gene is placed operably under the control of a promoter that is repressed in the presence of the compound.

In a particularly preferred embodiment, the compound is glucose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

In a preferred embodiment, the compound is phosphate and the promoter is a PHO5 promoter (SEQ ID NO: 6).

In another embodiment, conditions sufficient to produce cell death by virtue of reporter gene expression comprise incubating the cells in an amount of a plurality of compounds sufficient to modulate expression of a gene selected from the group consisting of a reporter gene and a gene encoding a binding partner to the DNA-protein or protein-protein interaction.

In a preferred embodiment, the gene encoding a binding partner comprises a promoter that is regulated by the plurality of compounds thereby modulating expression of the reporter gene by virtue of modulating the amount of a binding partner that regulates reporter gene expression in the cell.

In another embodiment, the reporter gene comprises a promoter that is regulated by the plurality of compounds thereby modulating expression of the reporter gene in the cell.

In a preferred embodiment, the gene encoding a binding partner or the reporter gene comprises a promoter that is induced in the presence of a compound and repressed in the presence another compound of said plurality of compounds.

In a particularly preferred embodiment, a compound is glucose and another compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

In a preferred embodiment, the amount of one or more compounds sufficient to modulate expression of a binding partner to a DNA-protein or a protein-protein interaction is determined by a method comprising:
(a) culturing a cell that does not express a protein interacting partner of a DNA-protein interaction or expresses one protein interacting partner of a protein-protein interaction in the presence of the one or more compounds that modulates expression of the protein binding partner and in the absence of a peptide inhibitor of the DNA-protein or protein-protein interaction and determining the amount of the compound that reduces cell death under conditions sufficient to produce cell death by virtue of reporter gene expression; and
(b) culturing a cell that expresses a protein interacting partner of a DNA-protein interaction or expresses the proteins of a protein-protein interaction in the presence of the compound that modulates expression of the protein binding partner and in the absence of a peptide inhibitor of the DNA-protein or protein-protein interaction and determining the amount of the compound that enhances cell death under conditions sufficient to produce cell death by virtue of reporter gene expression,
(c) determining an amount of the substrate that reduces cell death at (a) and enhances cell death at (b).

In one exemplification, the present invention provides an improved reverse N-hybrid assay for identifying a peptide inhibitor of a DNA-protein or protein-protein interaction in a yeast cell comprising:
(a) determining an amount of uracil required to enhance cell death in a cell in the absence of a peptide inhibitor of the DNA-protein or protein-protein interaction wherein the cell is cultured under conditions sufficient to produce cell death; and
(b) determining an amount of uracil required to enhance cell survival in the presence of a peptide inhibitor of the DNA-protein or protein-protein interaction wherein the cell is cultured under conditions sufficient to produce cell death; and
(c) performing a reverse N-hybrid assay using an amount of uracil as determined at (a) and (b).

In a particularly preferred embodiment the present invention provides an improved reverse N-hybrid assay for identifying a peptide inhibitor of a DNA-protein or protein-protein interaction in a yeast cell comprising:
(a) determining an amount of glucose and galactose required to modulate URA3 reporter gene expression and enhance cell death in the absence of a peptide inhibitor of the DNA-protein or protein-protein interaction;
(b) determining an amount of glucose and galactose required to modulate URA3 reporter gene expression and enhance cell survival in the presence of a peptide inhibitor of the DNA-protein or protein-protein interaction; and
(c) performing a reverse N-hybrid assay using an amount of the substrate or modulator as determined at (a) and (b),
wherein expression of the URA3 reporter gene modulates cell survival by virtue of encoding a polypeptide that reduces cell growth or viability by converting 5-fluororotic acid to a cytostatic or cytotoxic product.

In a further particularly preferred embodiment, the present invention provides an improved reverse N-hybrid assay for identifying a peptide inhibitor of a DNA-protein or protein-protein interaction in a yeast cell comprising:
(a) determining an amount of uracil and an amount of 5-fluororotic acid required to enhance cell death in a cell in the absence of a peptide inhibitor of the DNA-protein or protein-protein interaction wherein the cell is cultured under conditions sufficient to produce cell death; and
(b) determining an amount of uracil and an amount of 5-fluororotic acid required to enhance cell survival in the presence of a peptide inhibitor of the DNA-protein or protein-protein interaction wherein the cell is cultured under conditions sufficient to produce cell death; and
(c) performing a reverse N-hybrid assay using an amount of uracil and an amount of 5-fluororotic acid as determined at (a) and (b).

In yet another particularly preferred embodiment the present invention provides an improved reverse N-hybrid assay for identifying a peptide inhibitor of a DNA-protein or protein-protein interaction in a yeast cell comprising:
(a) determining an amount of uracil and an amount of 5-fluororotic acid and an amount of cycloheximide required to enhance cell death in a cell in the absence of a peptide inhibitor of the DNA-protein or protein-protein interaction wherein the cell is cultured under conditions sufficient to produce cell death; and
(b) determining an amount of uracil and an amount of 5-fluororotic acid and an amount of cycloheximide required to enhance cell survival in the presence of a peptide inhibitor of the DNA-protein or protein-protein interaction wherein the cell is cultured under conditions sufficient to produce cell death; and
(c) performing a reverse N-hybrid assay using an amount of uracil and an amount of 5-fluororotic acid as determined at (a) and (b).

Another aspect of the invention provides an improved forward N-hybrid assay for identifying a heterologous peptide or protein capable of binding to the DNA or protein in a cell comprising:
(a) determining an amount of a modulator of a reporter gene required to inhibit cell growth and/or survival in the absence of a heterologous peptide or protein capable of binding to the DNA or protein in a cell;
(b) determining an amount of a modulator of a reporter gene required to enhance cell growth and/or survival in the presence of a heterologous peptide or protein capable of binding to the DNA or protein in a cell; and
(c) performing a forward N-hybrid assay using an amount of the modulator as determined at (a) and (b);
wherein expression of the reporter gene modulates cell survival by virtue of encoding a polypeptide required for cell growth and/or survival.

Preferably, the cell is a yeast cell.

In the context of a forward N-hybrid assay the term “background number of cells” shall be understood to refer to the number of cells that do not express an interacting protein of a protein-DNA interaction or express an interacting protein of a protein-protein interaction and are incapable of surviving in the presence of a substrate of the reporter gene.

In one embodiment, the background number of cells is determined using a method comprising
(a) culturing a known number of cells that do not express a protein binding partner of a protein-DNA interaction or expresses an interacting protein of a protein-protein interaction in the presence of a substrate of a reporter gene under conditions sufficient to produce cell growth and/or survival by virtue of reporter gene expression; and
(b) determining the number of cells that survive and/or grow wherein any cells that survive and/or grow in the presence of the substrate of the reporter gene are background.

In the context of a forward N-hybrid assay the term “plating efficiency” shall be taken to mean the number of cells that do not express an interacting protein of a protein-DNA interaction or express an interacting protein of a protein-protein interaction and are capable of surviving in the presence of the substrate of the reporter gene

In one embodiment, the plating efficiency is determined by a method comprising:
(a) culturing a known number of cells that express a protein binding partner of a protein-DNA interaction or express the proteins of a protein-protein interaction in the presence of a substrate of a reporter gene, under conditions sufficient to produce cell growth and/or survival by virtue of reporter gene expression; and
(b) determining the number of cells that survive and/or grow compared to the number of cells cultured wherein a decrease in the number of cells that survive and/or grow compared to the number of cells cultured indicates a reduction in plating efficiency.

In a preferred embodiment, conditions sufficient to produce cell growth and/or survival by virtue of reporter gene expression comprise incubating the cells in an amount of a compound sufficient to modulate expression of a gene selected from the group consisting of a reporter gene and a gene encoding a binding partner to the DNA-protein or protein-protein interaction.

Preferably, the gene encoding a binding partner comprises a promoter that is regulated by the compound thereby modulating expression of the reporter gene by virtue of modulating the amount of a binding partner that regulates reporter gene expression in the cell.

In one embodiment, the reporter gene comprises a promoter that is regulated by the compound thereby modulating expression of the reporter gene in the cell.

Preferably, the gene encoding a binding partner or the reporter gene comprises a promoter that is induced in the presence of the compound.

In one embodiment, the compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

In another embodiment, the gene encoding a binding partner or the reporter gene comprises a promoter that is repressed in the presence of the compound. Preferably, the compound is glucose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

In another embodiment, the compound is phosphate and the promoter is a PHO5 promoter (SEQ ID NO: 6).

In one embodiment, conditions sufficient to produce cell growth and/or survival by virtue of reporter gene expression comprise incubating the cells in an amount of a plurality of compounds sufficient to modulate expression of a gene selected from the group consisting of a reporter gene and a gene encoding a binding partner to the DNA-protein or protein-protein interaction.

Preferably, the gene encoding a binding partner comprises a promoter that is regulated by the plurality of compounds thereby modulating expression of the reporter gene by virtue of modulating the amount of a binding partner that regulates reporter gene expression in the cell.

In one embodiment, the reporter gene comprises a promoter that is regulated by the plurality of compounds thereby modulating expression of the reporter gene in the cell.

In one embodiment, the gene encoding a binding partner or the reporter gene comprises a promoter that is induced in the presence of a compound and repressed in the presence another compound of said plurality of compounds. Preferably, a compound is glucose and another compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

In one embodiment, the amount of one or more compounds sufficient to modulate expression of a binding partner to a DNA-protein or a protein-protein interaction is determined by a method comprising:
(a) culturing a cell that does not express a protein binding partner of a DNA-protein interaction or expresses a protein binding partner of a protein-protein interaction in the presence of the one or more compounds that modulate expression of the protein binding partner and determining the amount of the compound that suppresses cell growth and/or survival under conditions sufficient to produce cell growth and/or survival by virtue of reporter gene expression; and
(b) culturing a cell that expresses a protein binding partner of a DNA-protein interaction or expresses the proteins of a protein-protein interaction in the presence of the one or more compounds that modulate expression of the protein binding partner and determining the amount of the one or more compounds sufficient to produce cell growth and/or survival by virtue of reporter gene expression,
wherein an amount of the substrate that suppresses cell growth and/or survival at (a) and produces cell growth and/or survival at (b) is an amount of the compound that enhances expression of the protein binding partner thereby enhancing cell growth and/or survival in the presence of a DNA-protein or protein-protein interaction and does not compromise or inhibit cell survival in the absence of DNA-protein or protein-protein interaction.

In another aspect the present invention provides a cell produced by the method of the invention, and a cell when produced by the method of the present invention

In accordance with the preceding embodiments, it is preferred for the improved reverse N-hybrid assay or the improved forward N-hybrid assay to express the protein binding partners operably under control of independently regulatable promoters. Preferably, the promoters are different promoters. For example, the two interacting proteins may be expressed under control of two inducible and repressible promoters.

### BRIEF DESCRIPTION OF THE DRAWINGS

FIG. 1 is a photographic representation showing yeast HWU-minimal media plates supplemented with various concentrations of sugar (as shown) on which yeast expressing the E47 bait protein with no interacting partner were grown. As can be seen no colonies grew, suggesting that there was no auto-activation of the URA43 reporter gene by the E47 bait protein.

FIG. 2 is a photographic representation showing yeast HWU-minimal media plates supplemented with various concentrations of sugar (as shown) on which yeast expressing the E47 bait protein and the SCL prey protein were expressed. In the presence of galactose alone the URA3 reporter gene is activated and yeast colonies are observed. As glucose is added to the media there is an attenuation of the expression of the E47 and SCL proteins, as they are expressed by a GAL1 promoter. The reduction in expression of the interacting proteins results in a decrease in the expression of the URA3 reporter molecule. This is seen as a reduction in the number of colonies growing on plates with increasing concentrations of glucose.

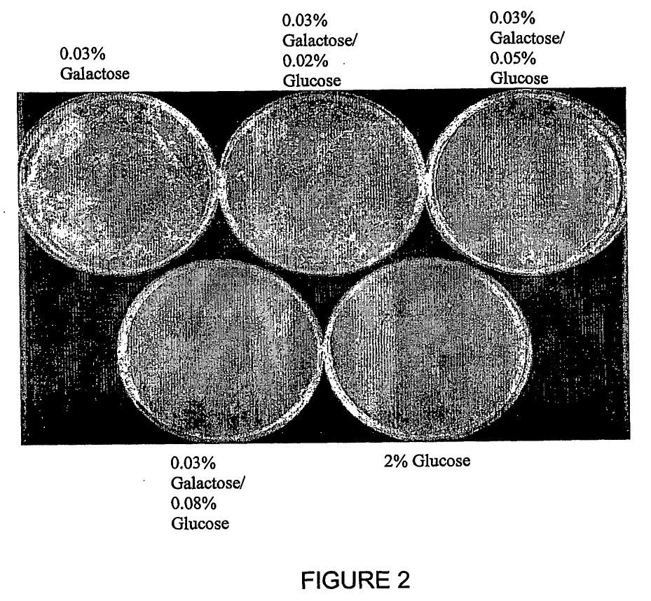

FIG. 3 is a photographic representation showing yeast HW-complete synthetic minimal media plates that have been supplemented with various sugars (as shown) in addition to 5′FOA (where shown) on which yeast expressing only the E47 protein were expressed. As the concentration of glucose is increased there is an obvious increase in the number of colonies able to grow on the selectable media.

FIG. 4 is a photographic representation showing yeast HW-complete synthetic minimal media plates that have been supplemented with various sugars (as shown) in addition to 5′FOA (where shown) on which yeast expressing both of the fusion partners is shown. As the concentration of glucose is increased there is an obvious increase in the number of colonies able to grow on the selectable media. However, on the plate where there a both glucose and galactose are present there are fewer colonies observed than the screen using only the E47 bait protein (as shown in FIG. 4). This suggests a reduction in the number of false positive colonies.

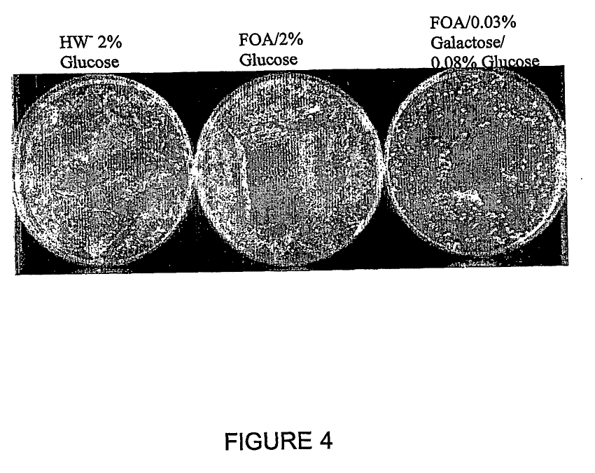

FIG. 5 is a photographic representation showing yeast HWU-media plates that have been supplemented with 0.02% galactose; 0.04% FOA; and 5 μg/ml cycloheximide and various concentrations of uracil (as indicated). Yeast plated express JUN1 and JUNZ (known interacting proteins) or JUN1, JUNZ and FOSZ (interacting proteins and an inhibitor of interaction). As the concentration of uracil is increased the number of colonies on the JUN1/JUNZ (19) plates increases (background) and the plating efficiency (as determined from the JUN1/JUNZ/FOSZ (19F) plates) is reduced. At lower concentrations of uracil the level of background is negligible and the plating efficiency is acceptable.

FIG. 6 is a tabular representation of the data derived from FIG. 5. Note that the level of background detected in the presence of low concentrations of uracil is 0 while plating efficiency is maintained at greater than 40%.

FIG. 7 is a graphical representation of HWU-plates supplemented with 0.02% galactose and 5 μg/ml cycloheximide and various concentrations of uracil and 5-FOA. Yeast plated express JUN1 and JUNZ (known interacting proteins) (19) or JUN1, JUNZ and FOSZ (interacting proteins and an inhibitor of interaction) (19F). As the levels of uracil are increased the amount of background is reduced while the plating efficiency is maintained.

FIG. 8 is a tabular representation of results of a study to determine the effect of uracil concentration and 5-FOA concentration on background and plating efficiency using yeast expressing JUN1 and JUNZ (known interacting proteins) (19) or JUN1, JUNZ and FOSZ (interacting proteins and an inhibitor of interaction) (19F) plated on HWU-plates supplemented with 0.02% galactose and 5 μg/ml cycloheximide and various concentrations of uracil and 5-FOA. Note the reduced background and maintained or enhanced plating efficiency with high concentrations of uracil and low concentrations of 5-FOA and with low concentrations of uracil and high concentrations of 5-FOA. The effect of these variables on increased plate loading (ie increased numbers of cells plated) is also shown.

FIG. 9A is a graphical representation showing HWU-plates supplemented with 0.2 mg/L uracil, 0.06% 5-FOA and various concentrations of cycloheximide. Left hand side of the plate has 10000 cells plated and right hand side of the plate has 100000 cells plated. Cells express JUN1 and JUNZ. Note that as the concentration of cycloheximide is decreased the number of cells growing (background) is increased.

FIG. 9B is a graphical representation showing HWU-plates supplemented with 0.2 mg/L uracil, 0.06% 5-FOA and various concentrations of cycloheximide. Left hand side of the plate has 10000 cells plated and right hand side of the plate has 100000 cells plated. Cells express JUN1, JUNZ and FOSZ. Note that as the concentration of cycloheximide is increased the number of cells growing (plating efficiency) is decreased.

FIG. 9C is a graphical representation showing HWU-plates supplemented with 0.5 mg/L uracil, 0.06% 5-FOA and various concentrations of cycloheximide. Left hand side of the plate has 10000 cells plated and right hand side of the plate has 100000 cells plated. Cells express JUN1 and JUNZ. Note that as the concentration of cycloheximide is decreased the number of cells growing (background) is increased. Note that the background is lower than the results shown in FIG. 9A

FIG. 9B is a graphical representation showing HWU-plates supplemented with 0.5 mg/L uracil, 0.06% 5-FOA and various concentrations of cycloheximide. Left hand side of the plate has 10000 cells plated and right hand side of the plate has 100000 cells plated. Cells express JUN1, JUNZ and FOSZ. Note that as the concentration of cycloheximide is increased the number of cells growing (plating efficiency) is decreased and that the plating efficiency is lower than the results shown in FIG. 9B.

FIG. 10 is a tabular representation of the results shown in FIGS. 9A-D. Note that negligible background levels were attained in the presence of 0.2 mg/L uracil, 0.06% 5-FOA and 5 μg/ml cycloheximide while acceptable plating efficiencies were maintained.

### DETAILED DESCRIPTION OF THE PREFERRED EMBODIMENTS

One aspect of the present invention provides an improved reverse N-hybrid assay for identifying a peptide inhibitor of a DNA-protein or protein-protein interaction in a cell comprising:
(a) determining an amount of a substrate or modulator of a reporter gene required to enhance cell death in the absence of a peptide inhibitor of the DNA-protein or protein-protein interaction; and/or
(b) determining an amount of a substrate or modulator of a reporter gene required to enhance cell survival in the presence of a peptide inhibitor of the DNA-protein or protein-protein interaction; and
(c) performing a reverse N-hybrid assay using an amount of the substrate or modulator as determined at (a) and (b),
wherein expression of the reporter gene modulates cell survival by virtue of encoding a polypeptide that reduces cell growth or viability by providing a target for a cytostatic or cytotoxic compound or by converting the substrate to a cytostatic or cytotoxic product.

As used herein, the term “a peptide inhibitor of a DNA-protein interaction or a protein-protein interaction” shall be taken to mean a peptide that is capable of binding to one or more binding partners on a DNA protein interaction or a protein-protein interaction and thereby disrupt said interaction. Preferably, the peptide inhibitor of a DNA-protein or protein-protein interaction is a heterologous peptide that is not endogenous to the cell or capable of modulating expression of the reporter gene.

In one embodiment, the present invention provides an improved reverse N-hybrid assay for identifying a peptide inhibitor of a DNA-protein or protein-protein interaction in a cell comprising:
(a) determining an amount of a substrate or modulator of a reporter gene required to enhance cell death in the absence of a peptide inhibitor of the DNA-protein or protein-protein interaction; and
(b) determining an amount of a substrate or modulator of a reporter gene required to enhance cell survival in the presence of a peptide inhibitor of the DNA-protein or protein-protein interaction; and
(c) performing a reverse N-hybrid assay using an amount of the substrate or modulator as determined at (a) and (b),
wherein expression of the reporter gene modulates cell survival by virtue of encoding a polypeptide that reduces cell growth or viability by providing a target for a cytostatic or cytotoxic compound or by converting the substrate to a cytostatic or cytotoxic product.

Preferably, the order in which determining an amount of a substrate or modulator of a reporter gene required to enhance cell death in the absence of a peptide inhibitor of the DNA-protein or protein-protein interaction and determining an amount of a substrate or modulator of a reporter gene required to enhance cell survival in the presence of a peptide inhibitor of the DNA-protein or protein-protein interaction are performed does not affect the working of the invention.

In another embodiment, the present invention provides an improved reverse N-hybrid assay for identifying a peptide inhibitor of a DNA-protein or protein-protein interaction in a cell comprising:
(a) determining an amount of a substrate or modulator of a reporter gene required to enhance cell death in the absence of a peptide inhibitor of the DNA-protein or protein-protein interaction; and
(b) determining an amount of a substrate or modulator of a reporter gene required to enhance cell survival in the presence of a peptide inhibitor of the DNA-protein or protein-protein interaction; or
(c) performing a reverse N-hybrid assay using an amount of the substrate or modulator as determined at (a) and (b),
wherein expression of the reporter gene modulates cell survival by virtue of encoding a polypeptide that reduces cell growth or viability by providing a target for a cytostatic or cytotoxic compound or by converting the substrate to a cytostatic or cytotoxic product.

In yet another embodiment, the present invention provides an improved reverse N-hybrid assay for identifying a peptide inhibitor of a DNA-protein or protein-protein interaction comprising the steps of:
(a) determining an amount of a substrate or modulator of a reporter gene required to enhance cell death in the absence of a peptide inhibitor of the DNA-protein or protein-protein interaction; and
(b) determining an amount of a substrate or modulator of a reporter gene required to enhance cell survival in the presence of a peptide inhibitor of the DNA-protein or protein-protein interaction; or
(c) performing a reverse N-hybrid assay using an amount of the substrate or modulator as determined at (a) and (b),
wherein expression of the reporter gene modulates cell survival by virtue of encoding a polypeptide that reduces cell growth or viability by providing a target for a cytostatic or cytotoxic compound or by converting the substrate to a cytostatic or cytotoxic product.

Reverse N-hybrid assays are known in the art and include, for example, a reverse one-hybrid assay, a reverse two-hybrid assay and a reverse split-two hybrid assay.

For example, a reverse two-hybrid assay is performed to identify a peptide that antagonizes or inhibits the interaction between a target protein or nucleic acid and another protein or nucleic acid. Accordingly, reverse N-hybrid screens are employed to identify agonist molecules. Reverse hybrid screens use a counter selectable reporter marker(s), such as for example the URA3 gene, the CYH2 gene or the LYS2 gene, to select against interactions between the target protein or nucleic acid and another protein or nucleic acid. Cell survival or cell growth is reduced or prevented in the presence of a toxigenic substrate of the counter selectable reporter gene product, which is converted by the counter selectable marker to a toxic compound, such as for example the CYH1 gene product which confers lethality in the presence of cycloheximide, and the URA3 gene product which confers lethality in the presence of 5-fluororotic acid. Accordingly, cells in which the interaction between the target protein and another protein or nucleic acid is blocked or inhibited survive in the presence of the substrate. This is because the counter selectable reporter molecule will not be expressed, and accordingly, the substrate will not be converted to a toxic product. Such a result suggests that a peptide is an inhibitor of the interaction between the target protein or nucleic acid and another protein or nucleic acid.

In one embodiment, the present invention provides an enhanced reverse 1-hybrid assay. A reverse 1-hybrid assay is useful for determining a peptide or polypeptide inhibitor of a DNA-protein interaction, for example for identifying a peptide that is capable of inhibiting the binding of a transcription factor to DNA. Such an assay generally comprises:

(i) optionally providing a cell that comprises a nucleic acid comprising a reporter gene (eg a detectable reporter gene and/or a selectable marker), said gene being positioned downstream of a promoter comprising a cis-acting element such that expression of said reporter gene is operably under the control of said promoter and wherein a protein binding partner of the DNA-protein interaction being assayed binds to said cis-acting element;
(ii) expressing a nucleic acid encoding a protein of the DNA-protein interaction that binds to a cis-acting element to activate expression of a reporter gene in a cell, wherein the reporter gene is placed in operable connection with a cis-acting element such that expression of said reporter gene is operably under the control of said promoter and wherein a protein binding partner of the DNA-protein interaction being assayed binds to said cis-acting element;
(iii) expressing a peptide in the cell; and
(iv) determining the effect of the expression of the peptide (iii) on the expression of the reporter gene.

Preferably, the reporter gene is a counter selectable marker (eg a URA3 gene) and the presence of a DNA-protein interaction is determined, for example, by culturing the cells in the presence of 5-FOA. Accordingly, only those cells that express an inhibitor of the DNA-protein interaction are capable of growing in the presence of 5-FOA.

Preferably, the reporter gene and/or the protein binding partner are placed operably in connection with an inducible and/or repressible promoter. More preferably, the reporter gene and/or the protein binding partner are placed operably in connection with a promoter that is both inducible and repressible, as described herein.

Optionally, the reverse 2-hybrid method also comprises isolating or identifying a peptide that inhibits a protein-protein interaction or nucleic acid encoding same.

In the case of a reverse two-hybrid assay, to determine a peptide or polypeptide inhibitor of a protein-protein interaction, such an assay generally comprises:

(i) optionally providing a cell that comprises a nucleic acid comprising a reporter gene (eg a detectable reporter gene and/or a selectable marker), said gene being positioned downstream of a promoter comprising a cis-acting element such that expression of said reporter gene is operably under the control of said promoter and wherein a protein binding partner of the DNA-protein interaction being assayed binds to said cis-acting element;
(ii) expressing a nucleic acid encoding a protein of the protein-protein interaction wherein the protein is fused to a protein domain that binds to a cis-acting element to activate expression of a reporter gene in a cell, wherein the reporter gene is placed in operable connection with a cis-acting element such that expression of said reporter gene is operably under the control of said promoter and wherein a protein binding partner of the DNA-protein interaction being assayed binds to said cis-acting element;
(iii) expressing another nucleic acid encoding a protein of the protein-protein interaction wherein the protein is fused to a protein domain that is a transcriptional activator;
(iii) expressing a peptide in the cell; and
(iv) determining the effect of the expression of the peptide (iii) on the expression of the reporter gene.

Preferably, the reporter gene is a counter selectable marker (eg a URA3 gene) and the presence of a DNA-protein interaction is determined, for example, by culturing the cells in the presence of 5-FOA. Accordingly, only those cells that express an inhibitor of the DNA-protein interaction are capable of growing in the presence of 5-FOA.

Preferably, the reporter gene and/or the gene encoding a protein binding partner and/or a gene encoding the peptide are placed operably in connection with an inducible and/or repressible promoter. More preferably, the reporter gene and/or the gene encoding a protein binding partner and/or a gene encoding the peptide are placed operably in connection with a promoter that is both inducible and repressible, as described herein.

Optionally, the reverse 2-hybrid method also comprises isolating or identifying a peptide that inhibits a protein-protein interaction or nucleic acid encoding same.

In a preferred embodiment, the method of the present invention is useful in a reverse two-hybrid screening process, such as, for example, essentially as described by Watt et al. (U.S. Ser. No. 09/227,652, incorporated herein by reference), for identifying an inhibitory peptide that partially or completely inhibits a target protein-protein interaction or DNA-protein interaction involving one or more protein binding partners. Such a method comprises:
(i) providing cells that each comprise: (a) a nucleic acid comprising a counter-selectable reporter gene encoding a polypeptide that is capable of reducing cell growth or viability by providing a target for a cytotoxic or cytostatic compound (eg., CYH2 gene that confers susceptibility to cycloheximide) or by converting a substrate to a cytotoxic or cytostatic product (eg., URA3 gene that converts 5-FOA to a toxic product), said gene being positioned downstream of a promoter comprising a cis-acting element such that expression of said gene is operably under the control of said promoter and wherein a protein binding partner of the protein-protein interaction or the DNA-protein interaction being assayed binds to said cis-acting element; and (b) nucleic acid selected from the group consisting of: (i) nucleic acid encoding a protein of the DNA-protein interaction that binds to said cis-acting element to activate expression of the counter-selectable reporter gene; and (ii) nucleic acids encoding two protein binding partners of the protein-protein interaction wherein a protein binding partner binds to the cis-acting element and the protein binding partners interact, said binding to the cis-acting element and said interaction being required to activate expression of the counter-selectable reporter gene;
(ii) transforming or transfecting the cells or a portion of the cells with a nucleic acid encoding at least one interacting partner of a DNA-protein or protein-protein interaction;
(iii) culturing the transformed or transfected cells for a time and under conditions sufficient for the protein binding partner(s) to activate expression of the counter-selectable reporter gene in the absence of inhibition of the protein-protein interaction or the DNA-protein interaction by an amino acid sequence encoded by the expression library;
(iv) culturing the transformed or transfected cells under conditions sufficient for an amino acid sequence (ie an interacting partner) to be expressed in each of said transformed or transfected cells or a proportion of said transformed or transfected cells;
(v) culturing the transformed or transfected cells in the presence of the substrate or the cytotoxic or cytostatic compound such that the expressed counter-selectable reporter gene reduces the growth or viability of the cells unless said expression is reduced by virtue of an amino acid sequence of the expression library inhibiting the target protein-protein interaction or DNA-protein interaction; and
(vi) selecting a cell having enhanced growth or viability compared to a cell that does not express the amino acid sequence of the expression library wherein the enhanced growth or viability is indicative of a partial or complete inhibition of the protein-protein interaction or a DNA-protein interaction by the amino acid sequence.

As will be apparent, the method of the present invention is useful in modulating the amount of the substrate of the counter selectable marker at (v) or modulating expression of an interacting partner by virtue of modulating culture conditions at (iv).

Preferably, wherein a protein-protein interaction is being assayed, the binding of the two protein binding partners reconstitutes a functional transcriptional regulatory protein, such as, for example, by virtue of the binding partners being expressed as fusion proteins wherein each fusion protein comprises a portion of a transcriptional regulatory protein that does not modulate transcription without the other portion (eg., a fusion protein comprising a transcriptional activator domain and a fusion protein comprising a DNA-binding domain).

As will be known to the skilled artisan, the reverse ‘n’-hybrid technique briefly described above is readily modified for use in 1-hybrid, 2-hybrid or 3-hybrid assays. Such systems are known in the art and reviewed, for example, Vidal and Legrain Nucl. Acids Res. 27: 919-929, 1999.

The present inventions further provides yeast strains that are preferably useful in the method of the present invention. For example, the method of the present invention is useful in a yeast cell comprising a genotype MATα, his3, trp1, ura3, 4 LexA-LEU2, lys2::3 cIop-LYS2, CANR, CYH2R, ade2::2 LexA-CYH2-ZEO, his5::1 LexA-URA3-G418, met15::Hygro. Alternatively, or in addition a yeast cell with the genotype MATα, his3, trp1, ura3, 4 LexA-LEU2, lys2::3 cIop-LYS2, CANR, CYH2R ade2::2 LexA-CYH2-ZEO, his5::2 LexA-URA3-G418, met15::Hygro is useful in the method of the present invention, as is a yeast cell with the genotype: MATα, his3, trp1, ura3, 4 LexA-LEU2, lys2::3 cIop-LYS2, CANR, CYH2R, ade2::2 LexA-CYH2-ZEO, his5::1 LexA-URA3-G418, met15::8LxLacZ-3cIGusA::ADE2. Another yeast cell useful for the performance of the present invention has a genotype comprising MATα, his3, trp1, ura3, 4 LexA-LEU2, lys2::3 cIop-LYS2, CANR, CYH2R, ade2::2 LexA-CYH2-ZEO, his5::2 LexA-URA3-G418, met15::8LxLacZ-3cIGusA::ADE2.

In an alternative embodiment, the antagonist is identified using a reverse split two hybrid screening process, such as, for example, essentially as described by Erickson et al. (WO95/26400, incorporated herein by reference), wherein a relay gene that is a negative regulator of transcription is employed to repress transcription of a positive readout reporter gene when the interacting proteins (ie., bait and prey) interact, such that reporter gene expression is only induced in the absence of the protein encoded by the relay gene product. Accordingly, such a method comprises:
(i) providing cells that each comprise: (a) a nucleic acid encoding a negative regulator of transcription (eg., Ga180 or mdm2 oncoprotein-encoding gene), said nucleic acid being positioned downstream of a promoter comprising a cis-acting element and wherein a protein binding partner of the protein-protein interaction or the DNA-protein interaction being assayed binds to said cis-acting element; (b) nucleic acid selected from the group consisting of: (i) nucleic acid encoding a protein of the DNA-protein interaction that binds to said cis-acting element to activate expression of the negative regulator of transcription; and (ii) nucleic acids encoding two protein binding partners of the protein-protein interaction wherein a protein binding partner binds to the cis-acting element and the protein binding partners interact, said binding to the cis-acting element and said interaction being required to activate expression of the negative regulator of transcription; and (c) nucleic acid comprising a positive reporter gene (eg., an antibiotic resistance gene, herbicide resistance gene, or other resistance gene) operably connected to a cis-acting element (eg., a GAL4 binding site capable of binding to Ga180, or Ga180, or the transactivation domain of p53 that binds to mdm2 oncoprotein) to which the negative regulator of transcription binds to thereby inhibit or repress expression of the positive reporter gene;
(ii) culturing the transformed or transfected cells for a time and under conditions sufficient for the protein binding partner(s) to activate expression of negative regulator of transcription in the absence of inhibition of the protein-protein interaction or the DNA-protein interaction by an amino acid sequence encoded by the expression library;
(iii) culturing the transformed or transfected cells under conditions sufficient for an interacting partner to be expressed in each of said transformed or transfected cells or a proportion of said transformed or transfected cells;
(iv) culturing the transformed or transfected cells in the presence of a compound to which the positive reporter gene confers resistance on the cells such that the expressed negative regulator of transcription represses expression of the positive reporter gene thereby reducing the growth or viability of the cells unless said expression is reduced by virtue of an amino acid sequence of the expression library inhibiting the target protein-protein interaction or DNA-protein interaction; and
(vi) selecting a cell having enhanced growth or viability compared to a cell that does not express the amino acid sequence of the expression library wherein the enhanced growth or viability is indicative of a partial or complete inhibition of the protein-protein interaction or a DNA-protein interaction by the amino acid sequence.

In a preferred embodiment, the improved reverse N-hybrid assay is performed in a yeast cell.

In the context of a reverse N-hybrid the term “background number of cells” shall be taken to mean the number of cells that express the interacting protein/s of a protein-DNA interaction or the proteins of a protein-protein interaction and are capable of surviving in the absence of a peptide inhibitor of a DNA-protein or protein-protein interaction and in the presence of the substrate of the reporter gene.

Preferably, the background number of cells is determined using a method comprising
(a) culturing a known number of cells that expresses at least one interacting protein of a protein-DNA interaction or at least two proteins of a protein-protein interaction in the presence of a substrate of a reporter gene under conditions sufficient to produce cell death by virtue of reporter gene expression; and
(b) determining the number of cells that survive and/or grow wherein any cells that survive and/or grow in the presence of the substrate of the reporter gene are background.

In the context of a reverse N-hybrid assay the term “plating efficiency” shall be taken to mean the number of cells that express the interacting protein/s of a protein-DNA interaction or the proteins of a protein-protein interaction and are capable of surviving in the presence of a peptide inhibitor of a DNA-protein or protein-protein interaction and the substrate of the reporter gene.

Preferably, the plating efficiency is determined by a method comprising:
(a) culturing a known number of cells that express at least one interacting protein of a protein-DNA interaction and a peptide inhibitor of the protein-DNA interaction or at least two proteins of a protein-protein interaction and a peptide inhibitor of the protein interaction in the presence of a substrate of a reporter gene under conditions sufficient to produce cell death by virtue of reporter gene expression; and
(b) determining the number of cells that survive and/or grow compared to the number of cells cultured wherein a decrease in the number of cells that survive and/or grow compared to the number of cells cultured indicates a reduction in plating efficiency.

In one preferred embodiment, conditions sufficient to produce cell death by virtue of reporter gene expression comprise incubating the cells in an amount of a toxigenic substrate of the reporter gene under conditions sufficient for the reporter gene to be capable of being expressed in the cell.

In one embodiment, the cell is a yeast cell and the toxigenic substrate is 5-fluororotic acid (5-FOA) and the reporter gene is URA3.

In another embodiment, the cell is a yeast cell and the toxigenic substrate is cycloheximide and the reporter gene is CYH2.

In a further embodiment, the cell is a yeast cell and the toxigenic substrate is α-aminoapidate and the reporter gene is LYS2.

In a particularly preferred embodiment, conditions sufficient to produce cell death by virtue of reporter gene expression comprise incubating the cells in an amount of a plurality of toxigenic substrates of a plurality of reporter genes under conditions sufficient for each reporter gene to be capable of being expressed in the cell.

In one embodiment, the cell is a yeast cell and the plurality of reporter genes is selected from the group consisting of URA3, CYH2, LYS2 and wherein the plurality of toxigenic substrates is selected from the group consisting of 5-fluororotic acid, cycloheximide and α-aminoapidate.

In yet another embodiment conditions sufficient to produce cell death by virtue of reporter gene expression comprise incubating the cells in media lacking an amount of a compound required for cell survival and complemented by a product of reporter gene expression under conditions sufficient for the reporter gene to be capable of being expressed in the cell.

In a particularly preferred embodiment, the cell is a yeast cell and the compound required for cell survival is uracil and the reporter gene is URA3.

In another embodiment, the compound required for cell survival is selected from the group consisting of histidine, tryptophan, leucine and methionine and the reporter gene is selected from the group consisting of HIS3, HIS5, TRP1, LEU2 and MET15.

In a further embodiment, conditions sufficient to produce cell death by virtue of reporter gene expression comprise incubating the cells in an amount of a compound sufficient to modulate expression of a gene selected from the group consisting of a reporter gene and a gene encoding a binding partner to the DNA-protein or protein-protein interaction.

Accordingly, in one embodiment, the present invention directly modulates the expression of a reporter gene, for example by virtue of placing the reporter gene in operable connection with an inducible and/or repressible promoter.

In another embodiment, the present invention indirectly modulates the expression of the reporter gene by modulating a binding partner to the DNA-protein or protein-protein interaction. As expression of the reporter gene is dependent on the DNA-protein or protein-protein interaction, modulation of one or more of the binding partners also modulates expression of the reporter gene. Preferably, the binding partner is placed in operable connection with an inducible and/or repressible promoter. Accordingly, the level of the binding partner is modulated by inducing and/or repressing the promoter.

In the context of the present invention the term “promoter” is to be taken in its broadest context and includes the transcriptional regulatory sequences of a genomic gene, including the TATA box or initiator element, which is required for accurate transcription initiation, with or without additional regulatory elements (ie. upstream activating sequences, transcription factor binding sites, enhancers and silencers) which alter gene expression in response to developmental and/or external stimuli, or in a tissue specific manner. Promoters may also be lacking a TATA box motif, however comprise one or more “initiator elements” or, as in the case of yeast-derived promoter sequences, comprise one or more “upstream activator sequence” elements. Such upstream activator sequence elements may be derived from another source and fused to the promoter, thus forming a chimeric promoter, or alternatively may form a part of the native promoter.

In the present context, the term “promoter” is also used to describe a recombinant, synthetic or fusion molecule, or derivative which confers, activates or enhances the expression of a nucleic acid molecule to which it is operably linked, and which encodes the peptide or protein. Preferred promoters can contain additional copies of one or more specific regulatory elements to further enhance expression and/or alter the spatial expression and/or temporal expression of said nucleic acid molecule.

Placing a nucleic acid molecule “in operable connection with”, ie. under the regulatory control of, a promoter sequence means positioning said molecule such that expression is controlled by the promoter sequence. Promoters are generally positioned 5′ (upstream) to the coding sequence that they control. Although some promoter system may be used to induce expression of multiple nucleic acids, such as for example, the GAL1/GAL10 promoter, wherein the promoter is able to induce expression of nucleic acids both upstream (3′) and downstream (5′) of the promoter sequence. To construct heterologous promoter/structural gene combinations, it is generally preferred to position the promoter at a distance from the gene transcription start site that is approximately the same as the distance between that promoter and the gene it controls in its natural setting, ie., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, ie., the gene from which it is derived. Again, as is known in the art, some variation in this distance can also occur.

In one embodiment, an inducible promoter is a chemically inducible promoter. In another embodiment, a repressible promoter is a chemically repressible promoter. In a still further embodiment, a promoter that is both inducible and repressible is a chemically regulated promoter.

A promoter that is “chemically regulated” or “chemically inducible” or “chemically repressible” or “regulatable” is a promoter from which expression levels may be altered or controlled through the addition of a compound to the media in which the cell expressing said nucleic acid is growing, wherein the compound is a synthetic or a naturally-occurring compound, such as, for example, a chemical compound selected from the group consisting of an amino acid, a metal ion, a sugar (eg., monosaccharide or disaccharide or polysaccharide), a salt, and a phosphate ion or salt thereof. Accordingly, by changing the concentration of a compound to which a chemically regulated promoter is exposed the level of expression of a gene to which said promoter is placed in operable connection is modulated.

In one embodiment, a chemical modulator (ie a compound) modulates the level of expression activity of a chemically inducible promoter through direct interaction with one or more components of the transcriptional machinery allowing these components to bind to sequences in the promoter that allow expression of a nucleic acid placed in operable connection with said promoter. In another embodiment, a chemical modulator inhibits the binding of an inhibitory protein or peptide to a component of the transcriptional machinery, thus allowing said component/s to induce expression. On the other hand, a chemical modulator may directly interact with one or more components of the transcriptional machinery and inhibit the interaction of said component/s with a promoter, consequently preventing the expression of a nucleic acid placed in operable connection with said promoter.

In one embodiment, a regulatable promoter is used to reduce or prevent expression under conditions wherein expression is undesirable. Accordingly a compound may be added to the media in which a cell is growing, said compound suppressing basal levels of expression of a nucleic acid in operable connection with a regulatable promoter contained within said cell. Suppression of basal expression levels is preferably useful in an assay that directly measures gene expression levels, or those that utilise a positive or negative selectable marker gene to select for a cell displaying a desirable phenotype, eg a forward or reverse N-hybrid screen).

In one embodiment, a regulatable promoter is used to reduce or prevent basal levels of expression in an assay that is used to measure the change in gene expression in a cell using a reporter gene. Preferably, basal levels of reporter gene expression are at least constant, and more preferably undetectable, so as to allow for ease of detection of a change of expression of said reporter gene in response to a stimulus (ega DNA-protein interaction or a protein-protein interaction). As used herein the term “stimulus” shall be taken to mean a factor that is able directly modulate the expression of a reporter molecule, or a factor that modulates a cellular process that in turn activates a reporter molecule. Assays that directly measure the expression of a reporter molecule are particularly useful in identifying factors that are able to modulate a signal transduction pathway, identifying factors that are able to directly bind to promoters or other nucleic acid sequences, identifying factors that are able to antagonise transcriptional or translational repressors, wherein the expression of a reporter gene is induced in response to a stimulus. Particularly useful reporter genes for use in such assay systems include a gene selected from the group consisting of β-galactosidase, chloramphenicol acetylase, luciferase, green fluorescent protein, a mutant of green fluorescent protein with a red shifted emission spectrum, a mutant of green fluorescent protein with a blue shifted emission spectrum or a mutant of green fluorescent protein with a yellow shifted emission spectrum.

In another embodiment, a regulatable promoter is used to reduce or prevent expression of a positive selectable marker, wherein expression of said positive selectable marker confers resistance to an antibiotic or a capacity to grow in conditions in which the cell is normally auxotrophic. Such systems are useful in the detection of an event or stimulus that activates expression of a positive selectable marker, as only those cells expressing said positive selectable marker are able to grow in selectable media. Accordingly, it is preferred that the expression level of a selectable marker is suppressed in those cells in which an event or stimulus has not induced said marker gene. Any basal or background expression of said selectable marker will lead to the identification of false positives, or cells in which the selectable marker gene has been expressed, but wherein the expression is not a result of induction by a stimulus. Assays in which the suppression of basal expression of a positive selectable marker include any of the N-hybrid systems known in the art and/or described herein. Preferred selectable markers include a gene selected from the group consisting of HIS3, HIS5, LEU2, LYS2 and URA3, all of which allow auxotrophic cells to grow in a culture medium lacking the appropriate amino acids and amp^{r} gene, kan^{r} gene, zeo^{r} gene, blas^{r} gene, and neo^{r} gene all of which confer resistance to one or more antibiotics.

Preferred assays in which the suppression of basal expression of a positive selectable marker is a forward N-hybrid assay. A forward N-hybrid assay is of use in identifying a protein or peptide that is able to interact with a particular protein or nucleic acid. A forward N-hybrid assay relies upon reporter molecules that are induced by a protein-protein or protein-nucleic acid interaction. Accordingly, “leaky” expression of a reporter molecule leads to the detection of false positives, or the ability of cells in which no interaction has occurred to grow under selectable conditions.

In yet another embodiment a regulatable promoter may be used to reduce or prevent expression of a counter selectable marker, wherein background expression is sufficient to confer lethality on a cell employed in an assay before expression is required to be induced. Such selectable markers are of particular use in identifying factors that are able to block an event from occurring, such as for example a protein-protein interaction, or a protein-nucleic acid interaction. In such assays the counter-selectable marker is expressed when the event to be blocked does occur, consequently those cells that antagonise said event do not express the counter-selectable marker, and as such do not die and are easily identified. Preferred counter selectable markers include a gene selected from the group consisting of the URA3 gene, which when expressed in the presence of 5′ fluorotic acid (FOA) produces a toxic product, the CYH2 gene which produces a toxic product in the presence of cyclohexamide, and the LYS2 gene.

Preferred assays in which the suppression of basal expression of a counter selectable marker is a reverse N-hybrid assay. A reverse N-hybrid assay is of use in identifying a protein or peptide that is able to block the interaction between a particular protein and another protein or a nucleic acid. A reverse N-hybrid assay relies upon negative selectable markers that are induced by a protein-protein or protein-nucleic acid interaction. The expression of a protein that blocks an interaction between a particular protein and another protein or nucleic acid suppresses the expression of a negative selectable marker. As expression of a negative reporter molecule leads to the death of a cell expressing said negative reporter molecule, basal expression levels of such a negative selectable marker leads to death of a cell expressing an antagonist of an interaction between a particular protein and another protein or a nucleic acid. This is particularly prevalent when detecting inhibitor proteins that interact with low affinity.

In a related embodiment, a regulatable promoter is used to enhance expression under conditions wherein expression is desired. Accordingly, a compound may be added to the media in which a cell is growing that enhances the expression of a nucleic acid that is placed in operable connection with a regulatable promoter contained within said cell. This is useful when basal levels of expression of a polypeptide are not sufficient to produce a desired outcome. In such cases it is necessary to induce the expression activity of a promoter placed in operable connection with a nucleic acid encoding said polypeptide.

In one embodiment, a regulatable promoter is used to enhance the expression of a peptide, polypeptide or protein of interest.

In a preferred embodiment, a regulatable promoter is used to enhance expression of polypeptides of interest in a forward or reverse N-hybrid assay. Polypeptides that may be expressed include a protein or peptide of interest fused to a DNA binding domain and/or a second protein or peptide of interest fused to an activation domain and/or one or more selectable markers or counter selectable markers. A chemically inducible promoter is used to control the expression of any or all of these proteins. In this way a N-hybrid system may be induced to express a bait protein and or a prey protein at a time that is appropriate for the screening method. Accordingly, when a reverse N-hybrid assay is to be used, it is preferred that putative inhibitors of a protein interaction are expressed prior to the expression of any interacting proteins. This is because, the interaction of said interacting proteins induces counter-selectable markers that result in toxicity to the cell. Accordingly the use of a regulatable promoter will allow the expression of any putative inhibitors in a cell prior to addition of a chemical compound to the media in which said cell is growing, resulting in expression of the interacting proteins.

In a preferred embodiment, a regulatable promoter for use in the present invention contains one or more specific regulatory elements to enhance or suppress expression of the gene. For example, regulatory elements that facilitate the expression of a gene by galactose, heavy metal ions, ethanol, arabinose, or copper may be placed adjacent to a heterologous promoter sequence driving expression of a gene. Alternatively a promoter that is inducible by galactose, heavy metal ions, ethanol, arabinose, or copper may be placed in operable connection with a gene.

In a preferred embodiment, a regulatable promoter for use in the present invention is induced by contacting said promoter with a chemical compound. Examples of chemically inducible promoters are the bacterial tac and lacUV5 promoters, which are induced by IPTG; the yeast copper inducible metallothionein promoter (cmt), which is induced by copper; the yeast alcohol dehydrogenase (ADH1) promoter, which is inducible with methanol; the CUP1 copper inducible promoter; the auxin inducible plant promoters P1 and P2; and the mammalian and insect metalothionein promoters, which are induced by metal ions, such as copper sulfate.

In another embodiment, regulatory elements that repress the expression of a gene in the presence of glucose, tryptophan, thiamine or phosphate may be placed adjacent to a heterologous promoter sequence driving expression of a gene. Alternatively, a gene may be placed in operable connection with a promoter that is repressed in the presence of glucose, tryptophan, thiamine or phosphate.

In a preferred embodiment, a regulatable promoter for use in the present invention is repressed by contacting said promoter with a chemical compound. Examples of chemically repressible promoters are the bacterial grg-1 promoter, which is repressed by glucose; the yeast fbp (fructose bisphosphate) promoter, which is repressed with glucose; the vbsO gene promoter, which is repressible with iron; and the bacterial trp promoters which are repressible with tryptophan.

In a preferred embodiment, a regulatable promoter comprises a binding site for one or more trans-acting regulatory proteins that activate or repress the activity of the promoter. Such binding sites may be endogenous to the native promoter, or derived from another source and inserted into the native promoter to produce a chimeric promoter construct.

In one embodiment, a binding site included in a regulatable promoter includes a binding site for a trans-acting regulatory protein selected from the group consisting of a GAL4 binding domain (SEQ ID NO: 7), a LexA operator sequence (SEQ ID NO: 8) or a cI operator sequence (SEQ ID NO: 9). Several other binding sites for trans-acting regulator proteins are known in the art and are commercially available, for example from Clontech Laboratories (Palo Alto, Calif., USA).

A regulatable promoter comprising a binding site for a trans-acting regulatory protein is of particular use when placed in operable connection with a counter-selectable marker gene. Expression of a regulatable promoter may then be suppressed so as to reduce background or “leaky” expression of said counter-selectable marker, thus reducing the number of false negative results. An advantage of a regulatable promoter comprising a binding site for a trans-acting regulatory protein when placed in operable connection with a counter-selectable marker gene is that said counter-selectable marker gene is induced by binding of a trans-acting regulatory protein to a binding site for said trans-acting regulatory protein.

In a preferred embodiment, a regulatable promoter of use in the present invention is inducible by a first chemical and repressible by a second chemical. Accordingly the expression of a nucleic acid placed in operable connection with such a regulatable promoter may be enhanced by the addition a first chemical. Alternatively, the expression may be suppressed by the addition of a second chemical. These promoters are particularly contemplated because the level of expression conferred by such a promoter may be limited to a desired level by adding combinations of both a first and a second chemical.

In a particularly preferred embodiment a regulatable promoter that is inducible by a first chemical and repressible by a second chemical is a promoter selected from the group consisting of, GAL1/GAL10 (SEQ ID NO: 1), GAL2 (SEQ ID NO: 2), GAL4 (SEQ ID NO: 3), GAL7 (SEQ ID NO: 4), and MEL1 (SEQ ID NO: 5).

In a preferred embodiment, the present invention provides a method of regulating the expression of a gene that is operably connected to a galactose-inducible promoter, said method comprising incubating the cell in an amount of glucose sufficient to reduce or prevent expression under conditions wherein expression is undesirable and/or an amount of galactose sufficient to increase or enhance expression wherein expression is desirable.

In a preferred embodiment, the gene encoding a binding partner comprises a promoter that is regulatable by the compound thereby modulating expression of the reporter gene by virtue of modulating the amount of a binding partner that regulates reporter gene expression in the cell. In one embodiment, the gene encoding a binding partner is placed in operable connection with a promoter that is regulated by the compound thereby modulating expression of the reporter gene in the cell.

In another embodiment, the reporter gene comprises a promoter that is regulated by the compound thereby modulating expression of the reporter gene in the cell. In one embodiment, the reporter gene is placed in operable connection with a promoter that is regulated by the compound thereby modulating expression of the reporter gene in the cell.

In a preferred embodiment, the gene encoding a binding partner or the reporter gene comprises a promoter that is induced in the presence of the compound. In one embodiment, the gene encoding a binding partner or the reporter gene is placed in operable connection with a promoter that is induced by the compound thereby modulating expression of the reporter gene in the cell.

In a particularly preferred embodiment the compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5). In another embodiment, the promoter is a promoter at least about 80% identical to a promoter selected form the group consisting of GAL1/GAL10 (SEQ ID NO: 1), GAL2 (SEQ ID NO: 2), GAL4 (SEQ ID NO: 3), GAL7 (SEQ ID NO: 4), and MEL1 (SEQ ID NO: 5), wherein the promoter is induced in the presence of galactose. More preferably, the degree of identity is about 85% to 90%, more preferably 90% to 95% and even more preferably 95% to 99% identity.

In determining whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT program or other appropriate program of the Computer Genetics Group, Inc., University Research Park, Madison, Wis., United States of America (Devereaux et al, Nucl. Acids Res. 12, 387-395, 1984).

In another preferred embodiment, the gene encoding a binding partner or the reporter gene is placed operably under the control of a promoter that is repressed in the presence of the compound. In one embodiment, the gene encoding a binding partner or the reporter gene is placed in operable connection with a promoter that is repressed by the compound thereby modulating expression of the reporter gene in the cell.

In a particularly preferred embodiment, the compound is glucose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5). In another embodiment, the promoter is a promoter at least about 80% identical to a promoter selected form the group consisting of GAL1/GAL10 (SEQ ID NO: 1), GAL2 (SEQ ID NO: 2), GAL4 (SEQ ID NO: 3), GAL7 (SEQ ID NO: 4), and MEL1 (SEQ ID NO: 5), wherein the promoter is repressed in the presence of glucose. More preferably, the degree of identity is about 85% to 90%, more preferably 90% to 95% and even more preferably 95% to 99% identity.

In a preferred embodiment, the compound is phosphate and the promoter is a PHO5 promoter (SEQ ID NO: 6). In another embodiment, the promoter is a promoter that is at least about 80% identical to a PHO5 promoter (SEQ ID NO: 6) wherein the promoter is repressed in the presence of phosphate. More preferably, the degree of identity is about 85% to 90%, more preferably 90% to 95% and even more preferably 95% to 99% identity.

In another embodiment, conditions sufficient to produce cell death by virtue of reporter gene expression comprise incubating the cells in an amount of a plurality of compounds sufficient to modulate expression of a gene selected from the group consisting of a reporter gene and a gene encoding a binding partner to the DNA-protein or protein-protein interaction.

In a preferred embodiment, the gene encoding a binding partner comprises a promoter that is regulated by the plurality of compounds thereby modulating expression of the reporter gene by virtue of modulating the amount of a binding partner that regulates reporter gene expression in the cell.

In another embodiment, the reporter gene comprises a promoter that is regulated by the plurality of compounds thereby modulating expression of the reporter gene in the cell.

In a preferred embodiment, the gene encoding a binding partner or the reporter gene comprises a promoter that is induced in the presence of a compound and repressed in the presence another compound of said plurality of compounds.

In a particularly preferred embodiment, a compound is glucose and another compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5). In another embodiment, the promoter is a promoter at least about 80% identical to a promoter selected form the group consisting of GAL1/GAL10 (SEQ ID NO: 1), GAL2 (SEQ ID NO: 2), GAL4 (SEQ ID NO: 3), GAL7 (SEQ ID NO: 4), and MEL1 (SEQ ID NO: 5), wherein the promoter is repressed in the presence of glucose and induced in the presence of galactose. More preferably, the degree of identity is about 85% to 90%, more preferably 90% to 95% and even more preferably 95% to 99% identity.

In a preferred embodiment, the amount of one or more compounds sufficient to modulate expression of a binding partner to a DNA-protein or a protein-protein interaction is determined by a method comprising:
(a) culturing a cell that does not express a protein interacting partner of a DNA-protein interaction or expresses one protein interacting partner of a protein-protein interaction in the presence of the one or more compounds that modulates expression of the protein binding partner and in the absence of a peptide inhibitor of the DNA-protein or protein-protein interaction and determining the amount of the compound that reduces cell death under conditions sufficient to produce cell death by virtue of reporter gene expression; and
(b) culturing a cell that expresses a protein interacting partner of a DNA-protein interaction or expresses the proteins of a protein-protein interaction in the presence of the compound that modulates expression of the protein binding partner and in the absence of a peptide inhibitor of the DNA-protein or protein-protein interaction and determining the amount of the compound that enhances cell death under conditions sufficient to produce cell death by virtue of reporter gene expression,
(c) determining an amount of the substrate that reduces cell death at (a) and enhances cell death at (b).

In one exemplification, the present invention provides an improved reverse N-hybrid assay for identifying a peptide inhibitor of a DNA-protein or protein-protein interaction in a yeast cell comprising:
(a) determining an amount of uracil required to enhance cell death in a cell in the absence of a peptide inhibitor of the DNA-protein or protein-protein interaction wherein the cell is cultured under conditions sufficient to produce cell death; and
(b) determining an amount of uracil required to enhance cell survival in the presence of a peptide inhibitor of the DNA-protein or protein-protein interaction wherein the cell is cultured under conditions sufficient to produce cell death; and
(c) performing a reverse N-hybrid assay using an amount of uracil as determined at (a) and (b).

In a particularly preferred embodiment the present invention provides an improved reverse N-hybrid assay for identifying a peptide inhibitor of a DNA-protein or protein-protein interaction in a yeast cell comprising:
(a) determining an amount of glucose and galactose required to modulate URA3 reporter gene expression and enhance cell death in the absence of a peptide inhibitor of the DNA-protein or protein-protein interaction;
(b) determining an amount of glucose and galactose required to modulate URA3 reporter gene expression and enhance cell survival in the presence of a peptide inhibitor of the DNA-protein or protein-protein interaction; and
(c) performing a reverse N-hybrid assay using an amount of the substrate or modulator as determined at (a) and (b),
wherein expression of the URA3 reporter gene modulates cell survival by virtue of encoding a polypeptide that reduces cell growth or viability by converting 5-fluororotic acid to a cytostatic or cytotoxic product.

In a further particularly preferred embodiment, the present invention provides an improved reverse N-hybrid assay for identifying a peptide inhibitor of a DNA-protein or protein-protein interaction in a yeast cell comprising:
(a) determining an amount of uracil and an amount of 5-fluororotic acid required to enhance cell death in a cell in the absence of a peptide inhibitor of the DNA-protein or protein-protein interaction wherein the cell is cultured under conditions sufficient to produce cell death; and
(b) determining an amount of uracil and an amount of 5-fluororotic acid required to enhance cell survival in the presence of a peptide inhibitor of the DNA-protein or protein-protein interaction wherein the cell is cultured under conditions sufficient to produce cell death; and
(c) performing a reverse N-hybrid assay using an amount of uracil and an amount of 5-fluororotic acid as determined at (a) and (b).

In yet another preferred embodiment the present invention provides an improved reverse N-hybrid assay for identifying a peptide inhibitor of a DNA-protein or protein-protein interaction in a yeast cell comprising:
(a) determining an amount of 5-fluororotic acid and an amount of cycloheximide required to enhance cell death in a cell in the absence of a peptide inhibitor of the DNA-protein or protein-protein interaction wherein the cell is cultured under conditions sufficient to produce cell death; and
(b) determining an amount of 5-fluororotic acid and an amount of cycloheximide required to enhance cell survival in the presence of a peptide inhibitor of the DNA-protein or protein-protein interaction wherein the cell is cultured under conditions sufficient to produce cell death; and
(c) performing a reverse N-hybrid assay using an amount of 5-fluororotic acid and an amount of cycloheximide as determined at (a) and (b).

In yet another particularly preferred embodiment the present invention provides an improved reverse N-hybrid assay for identifying a peptide inhibitor of a DNA-protein or protein-protein interaction in a yeast cell comprising:
(a) determining an amount of uracil and an amount of 5-fluororotic acid and an amount of cycloheximide required to enhance cell death in a cell in the absence of a peptide inhibitor of the DNA-protein or protein-protein interaction wherein the cell is cultured under conditions sufficient to produce cell death; and
(b) determining an amount of uracil and an amount of 5-fluororotic acid and an amount of cycloheximide required to enhance cell survival in the presence of a peptide inhibitor of the DNA-protein or protein-protein interaction wherein the cell is cultured under conditions sufficient to produce cell death; and
(c) performing a reverse N-hybrid assay using an amount of uracil and an amount of 5-fluororotic acid and an amount of cycloheximide as determined at (a) and (b).

In another aspect, the present invention provides an improved forward N-hybrid assay for identifying a heterologous peptide or protein capable of binding to the DNA or protein in a cell comprising:
(a) determining an amount of a modulator of a reporter gene required to enhance cell death in the absence of a heterologous peptide or protein capable of binding to the DNA or protein in a cell; and/or
(b) determining an amount of a modulator of a reporter gene required to enhance cell survival in the presence of a heterologous peptide or protein capable of binding to the DNA or protein in a cell; and
(c) performing a forward N-hybrid assay using an amount of the modulator as determined at (a) and (b);
   wherein expression of the reporter gene modulates cell survival by virtue of encoding a polypeptide that reduces cell growth or viability by providing a target for a cytostatic or cytotoxic compound or by converting a substrate to a cytostatic or cytotoxic product.

Forward N-hybrid methods are known in the art and include for example a one hybrid assay. A one hybrid assay is useful for the identification of nucleic acids that encode peptides having a conformation capable of binding to a DNA sequence. The one-hybrid assay, as described in Chong and Mandel (In: Bartel and Fields, The Yeast Two-Hybrid System, New York, N.Y. pp 289-297, 1997) is used to determine those peptides able to bind to a target DNA sequence. In a standard one-hybrid technique the target nucleotide sequence is incorporated into the promoter region of a reporter gene(s). The peptide of interest is expressed in such a manner that it forms a fusion protein with a transcriptional activation domain (for example from the GAL4 protein, the LexA protein, or the mouse NF κB protein). The transcriptional activation domain is recruited to the promoter through a functional interaction between the expressed peptide and the target nucleotide sequence. The transcriptional activation domain subsequently interacts with the basal transcriptional machinery of the cell, activating expression of the reporter genes.

Alternatively, a peptide or polypeptide is identified that is able to bind a target protein or peptide using the two-hybrid assay described in U.S. Pat. No. 6,316,223 to Payan et al and Bartel and Fields, The Yeast Two-Hybrid System, New York, N.Y., 1997 (both of which are incorporated herein by reference). The basic mechanism described requires that the binding partners are expressed as two distinct fusion proteins in an appropriate host cell, such as for example bacterial cells, yeast cells, and mammalian cells. A standard two-hybrid screen uses, a first fusion protein consisting of a DNA binding domain fused to the target protein, and a second fusion protein consists of a transcriptional activation domain fused to another protein or peptide (eg a “test” protein or peptide). The DNA binding domain binds to an operator sequence which controls expression of one or more reporter genes. The transcriptional activation domain is recruited to the promoter through the functional interaction between the peptide or protein and the target protein. Subsequently, the transcriptional activation domain interacts with the basal transcription machinery of the cell, thereby activating expression of the reporter gene(s), the expression of which can be determined.

The three hybrid assay as described in Zhang et al (In: Bartel and Fields, The Yeast Two-Hybrid System, New York, N.Y. pp 289-297, 1997) (incorporated herein by reference) is used to determine those peptides that bind target RNA sequences. The described 3-hybrid technique comprises, a first fusion protein consists of a DNA binding domain which is fused to a known RNA binding protein, eg. the coat protein of bacteriophage MS2. An RNA hybrid molecule is also formed, consisting of a fusion between a RNA molecule known to bind the RNA binding protein, eg. MS2 binding sequences, and a target RNA binding sequence. A second fusion protein consists of a transcriptional activation domain fused to, for example, a peptide. The DNA binding domain of the first fusion protein binds to an operator sequence that controls expression of one or more reporter genes. The RNA fusion molecule is recruited to the first fusion protein through the functional interaction between the RNA binding protein and the RNA molecule known to interact with said RNA binding protein. The transcriptional activation domain is recruited to the promoter of one or more reporter molecules through functional interaction between the target RNA sequence of the peptide.

The present invention also provides yeast cells that are preferably useful in performing an enhanced forward N-hybrid assay. For example, such a yeast cell comprises the genotype MATα, his3, trp1, ura3, 4 LexA-LEU2, lys2::3 clop-LYS2, CANR, met15::2LxURA3-G418. Alternatively, or in addition, a cell comprising the genotype MATα, his3, trp1, ura3, 4 LexA-LEU2, lys2::3 cIop-LYS2, CANR, met15::8LxURA3-G418 is useful in the method of the present invention as is a yeast cell with the genotype MATα, trpΔ1::hisG his3Δ200 leu2-3 lys2Δ201 ura3-52 met15::2LxURA3-G418. Another yeast cell useful in an enhanced forward N-hybrid assay comprises a genotype MATα, his3, trp1, ura3, 4 LexA-LEU2, lys2::3 cIop-LYS2, CANR, CYH2R, ade2::2 LexA-CYH2-ZEO, his5::2LexA-URA3-G418, met15::8LxLacZ-3cIGusA::ADE2.

The method of the present invention is useful for enhancing any of the assays described supra by, for example, reducing background and/or enhancing plating efficiency. Such a method is useful, for example, for increasing the number of cells screened in a N-hybrid screen.

In one embodiment, the present invention provides an improved forward N-hybrid assay for identifying a heterologous peptide or protein capable of binding to the DNA or protein in a cell comprising:
(a) determining an amount of a modulator of a reporter gene required to enhance cell death in the absence of a heterologous peptide or protein capable of binding to the DNA or protein in a cell; and
(b) determining an amount of a modulator of a reporter gene required to enhance cell death in the absence of a heterologous peptide or protein capable of binding to the DNA or protein in a cell; and
(c) performing a forward N-hybrid assay using an amount of the modulator as determined at (a) and (b);
   wherein expression of the reporter gene modulates cell survival by virtue of encoding a polypeptide that reduces cell growth or viability by providing a target for a cytostatic or cytotoxic compound or by converting a substrate to a cytostatic or cytotoxic product.

In the context of the present invention, determining an amount of a modulator of a reporter gene required to enhance cell death in the absence of a heterologous peptide or protein capable of binding to the DNA or protein in a cell and determining an amount of a modulator of a reporter gene required to enhance cell death in the absence of a heterologous peptide or protein capable of binding to the DNA or protein in a cell may be performed independently (wherein they may be performed in any order) or concurrently.

In one embodiment, the present invention provides an improved forward N-hybrid assay for identifying a heterologous peptide or protein capable of binding to the DNA or protein in a cell comprising:
(a) determining an amount of a modulator of a reporter gene required to enhance cell death in the absence of a heterologous peptide or protein capable of binding to the DNA or protein in a cell; or
(b) determining an amount of a modulator of a reporter gene required to enhance cell death in the absence of a heterologous peptide or protein capable of binding to the DNA or protein in a cell; and
(c) performing a forward N-hybrid assay using an amount of the modulator as determined at (a) and (b);
   wherein expression of the reporter gene modulates cell survival by virtue of encoding a polypeptide that reduces cell growth or viability by providing a target for a cytostatic or cytotoxic compound or by converting a substrate to a cytostatic or cytotoxic product.

In another embodiment, the present invention provides an improved forward N-hybrid assay for identifying a heterologous peptide or protein capable of binding to the DNA or protein in a cell comprising the steps of:
(a) determining an amount of a modulator of a reporter gene required to enhance cell death in the absence of a heterologous peptide or protein capable of binding to the DNA or protein in a cell; and
(b) determining an amount of a modulator of a reporter gene required to enhance cell death in the absence of a heterologous peptide or protein capable of binding to the DNA or protein in a cell; and
(c) performing a forward N-hybrid assay using an amount of the modulator as determined at (a) and (b);
   wherein expression of the reporter gene modulates cell survival by virtue of encoding a polypeptide that reduces cell growth or viability by providing a target for a cytostatic or cytotoxic compound or by converting a substrate to a cytostatic or cytotoxic product.

Preferably, the cell is a yeast cell.

In the context of a forward N-hybrid assay the term “background number of cells” shall be understood to refer to the number of cells that do not express an interacting protein of a protein-DNA interaction or express an interacting protein of a protein-protein interaction and are incapable of surviving in the presence of a substrate of the reporter gene.

In one embodiment, the background number of cells is determined using a method comprising
(a) culturing a known number of cells that do not express a protein binding partner of a protein-DNA interaction or expresses an interacting protein of a protein-protein interaction in the presence of a substrate of a reporter gene under conditions sufficient to produce cell death by virtue of reporter gene expression; and
(b) determining the number of cells that survive and/or grow wherein any cells that survive and/or grow in the presence of the substrate of the reporter gene are background.

In the context of a forward N-hybrid assay the term “plating efficiency” shall be taken to mean the number of cells that do not express an interacting protein of a protein-DNA interaction or express an interacting protein of a protein-protein interaction and are capable of surviving in the presence of the substrate of the reporter gene

In one embodiment, plating efficiency is determined by a method comprising:
(a) culturing a known number of cells that do not express a protein binding partner of a protein-DNA interaction or express a protein of a protein-protein interaction in the presence of a substrate of a reporter gene, under conditions sufficient to produce cell death by virtue of reporter gene expression; and
(b) determining the number of cells that survive and/or grow compared to the number of cells cultured wherein a decrease in the number of cells that survive and/or grow compared to the number of cells cultured indicates a reduction in plating efficiency.

In one embodiment, conditions sufficient to produce cell death by virtue of reporter gene expression comprise incubating the cells in an amount of a compound sufficient to modulate expression of a gene selected from the group consisting of a reporter gene and a gene encoding a binding partner to the DNA-protein or protein-protein interaction.

Preferably, the gene encoding a binding partner comprises a promoter that is regulated by the compound thereby modulating expression of the reporter gene by virtue of modulating the amount of a binding partner that regulates reporter gene expression in the cell.

In one embodiment, the reporter gene comprises a promoter that is regulated by the compound thereby modulating expression of the reporter gene in the cell.

In another embodiment, the gene encoding a binding partner or the reporter gene comprises a promoter that is induced in the presence of the compound. Preferably, the compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5). In another embodiment, the promoter is a promoter at least about 80% identical to a promoter selected form the group consisting of GAL1/GAL10 (SEQ ID NO: 1), GAL2 (SEQ ID NO: 2), GAL4 (SEQ ID NO: 3), GAL7 (SEQ ID NO: 4), and MEL1 (SEQ ID NO: 5), wherein the promoter is enhanced in the presence of galactose. More preferably, the degree of identity is about 85% to 90%, more preferably 90% to 95% and even more preferably 95% to 99% identity.

In yet another embodiment, the gene encoding a binding partner or the reporter gene comprises a promoter that is repressed in the presence of the compound. Preferably, the compound is glucose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5). In another embodiment, the promoter is a promoter at least about 80% identical to a promoter selected form the group consisting of GAL1/GAL10 (SEQ ID NO: 1), GAL2 (SEQ ID NO: 2), GALA (SEQ ID NO: 3), GAL7 (SEQ ID NO: 4), and MEL1 (SEQ ID NO: 5), wherein the promoter is repressed in the presence of glucose. More preferably, the degree of identity is about 85% to 90%, more preferably 90% to 95% and even more preferably 95% to 99% identity.

In another embodiment, the compound is phosphate and the promoter is a PHO5 promoter (SEQ ID NO: 6). In another embodiment, the promoter is a promoter at least about 80% identical to a PHO5 promoter, wherein the promoter is repressed in the presence of phosphate. More preferably, the degree of identity is about 85% to 90%, more preferably 90% to 95% and even more preferably 95% to 99% identity.

In a further embodiment, conditions sufficient to produce cell death by virtue of reporter gene expression comprise incubating the cells in an amount of a plurality of compounds sufficient to modulate expression of a gene selected from the group consisting of a reporter gene and a gene encoding a binding partner to the DNA-protein or protein-protein interaction. Preferably, the gene encoding a binding partner comprises a promoter that is regulated by the plurality of compounds thereby modulating expression of the reporter gene by virtue of modulating the amount of a binding partner that regulates reporter gene expression in the cell.

In one embodiment, the reporter gene comprises a promoter that is regulated by the plurality of compounds thereby modulating expression of the reporter gene in the cell.

In another embodiment, the gene encoding a binding partner or the reporter gene comprises a promoter that is induced in the presence of a compound and repressed in the presence another compound of said plurality of compounds. Preferably, a compound is glucose and another compound is galactose and the promoter is selected from the group consisting of a GAL1/GAL10 promoter (SEQ ID NO: 1), a GAL2 promoter (SEQ ID NO: 2), a GALA promoter (SEQ ID NO: 3), a GAL7 promoter (SEQ ID NO: 4) and a MEL1 promoter (SEQ ID NO: 5). In another embodiment, the promoter is a promoter at least about 80% identical to a promoter selected form the group consisting of GAL1/GAL10 (SEQ ID NO: 1), GAL2 (SEQ ID NO: 2), GAL4 (SEQ ID NO: 3), GAL7 (SEQ ID NO: 4), and MEL1 (SEQ ID NO: 5), wherein the promoter is repressed in the presence of glucose and enhanced in the presence of galactose. More preferably, the degree of identity is about 85% to 90%, more preferably 90% to 95% and even more preferably 95% to 99% identity.

In one embodiment, the amount of one or more compounds sufficient to modulate expression of a binding partner to a DNA-protein or a protein-protein interaction is determined by a method comprising:
(a) culturing a cell that does not express a protein binding partner of a DNA-protein interaction or expresses a protein binding partner of a protein-protein interaction in the presence of the one or more compounds that modulate expression of the protein binding partner and determining the amount of the compound that reduces cell death under conditions sufficient to produce cell death by virtue of reporter gene expression; and
(b) culturing a cell that expresses a protein binding partner of a DNA-protein interaction or expresses the proteins of a protein-protein interaction in the presence of the one or more compounds that modulate expression of the protein binding partner and determining the amount of the compound that enhances cell death by virtue of reporter gene expression,
wherein an amount of the substrate that suppresses cell death at (a) and does not suppress cell death at (b) is an amount of the compound that enhances expression of the protein binding partner thereby enhancing cell death in the presence of a DNA-protein or protein-protein interaction and does not compromise or inhibit cell survival in the absence of DNA-protein or protein-protein interaction.

The following invention is further described in the following non-limiting examples.

### Example 1

### Determining the Concentration of Glucose and Galactose so as to Reduce Background in a Reverse Two-Hybrid Screen

Prior to commencing a reverse two hybrid screen for inhibitors of the interaction between the polymyositis-scleroderma autoantigen (SCL) and basic helix-loop-helix transcription factor E47, it was necessary to determine the amount of glucose and galactose required to reduce background levels of auto-activation of the counter-selectable markers URA3 which is controlled by 2 Lex A operator sequences and CYH2 expression of which is also controlled by 2 Lex A operator sequences.

The nucleic acid encoding the SCL protein was cloned into the prey vector pJG4-5 (Clontech Laboratories Inc.) in operable connection with a nuclear localisation signal, and a B42 activation domain. The pJG4-5 vector also contains a nucleic acid encoding the TRP1 gene. The nucleic acid encoding the E47 protein was cloned into the bait vector pGILDA (Clontech Laboratories Inc.) in operable connection with the LexA DNA binding domain. The pGILDA vector also contains a nucleic acid encoding the HIS3 gene.

The pGil-E47 vector was transformed into the PRT 475 yeast strain (which has the phenotype MATα, his3, trp1, ura3, 4 LexA-LEU2, lys2::3 cIop-LYS2, CANR, CYH2R, ade2::2 LexA-CYH2-ZEO, met15::1 LexA-URA3-G418). The pJG-SCL vector was then transformed into the PRT 48 yeast strain (which has the phenotype MATα, his3, trp1, ura3, 6 LexA-LEU2, lys2::3 cIop-LYS2, CYH2R, ade2::G418-pZero-ADE2). These strains were then mated to allow for interaction of the protein binding partners, and diploids were selected on HW-complete synthetic minimal media supplemented with 2% (w/v) glucose.

In addition, the pGilda-E47 vector was transformed into the PRT475 strain of yeast, while the pJG4-5 vector with no insert was transformed into the PRT 48 strain of yeast. The transformed yeast were then mated and diploids were selected as before, and this formed the non-interacting control for determining the concentration of glucose and galactose required for an effective reverse two-hybrid assay.

Yeast containing the interacting proteins (pGil-E47/pJG-SCL) and yeast comprising non-interacting proteins (pGilda-E47/pJG4-5) were then grown on HWU-complete synthetic minimal media supplemented with one of the following combinations of sugars:

0.03% galactose
0.03% galactose/0.02% glucose
0.03% galactose/0.05% glucose
0.03% galactose/0.08% glucose
2% glucose

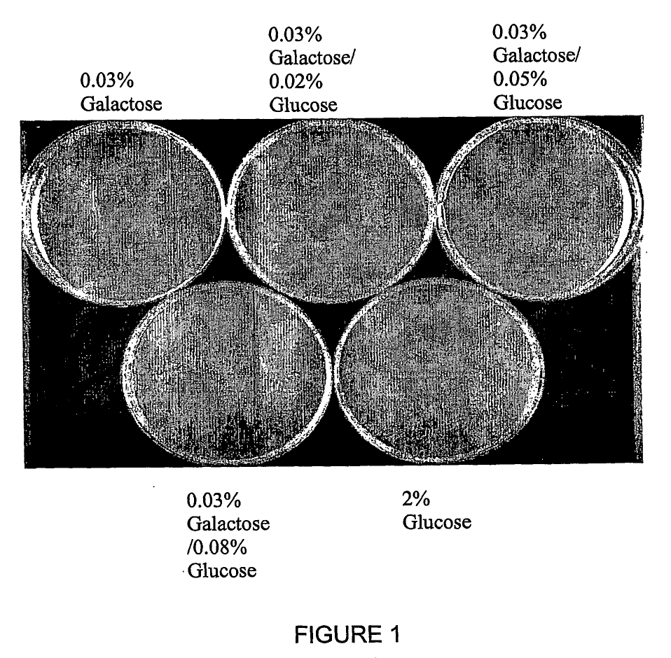

As can be seen in FIG. 1 the non-interacting control strain shows no auto-activation by the E47 bait protein. However addition of glucose to the media of the yeast comprising the interacting proteins shows a clear suppression of activation of the URA3 reporter gene. This is because expression of the E47 protein and the SCL protein are controlled by the GAL1 promoter. Expression of the GAL1 promoter is enhanced by the addition of galactose, and suppressed by the addition of glucose.

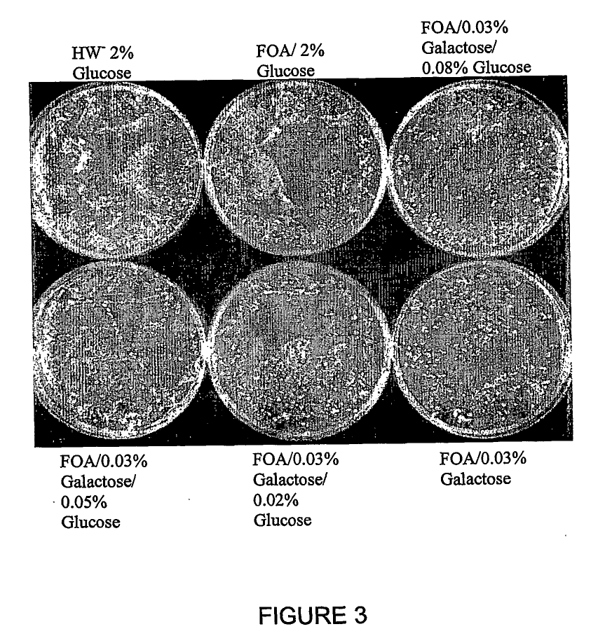

When the non-interacting control yeast were grown on negative selectable media (i.e. that containing 5′ FOA) glucose supplementation clearly increases the number of clones that are able to survive on such selectable media (FIG. 3). While this effect is also observed in the yeast expressing the interacting proteins, it is not to the same degree as that seen in the non-interacting controls. In the case of the SCL/E47 protein interaction it can clearly be seen that the combination of glucose and galactose will clearly reduce the background in a reverse two-hybrid assay. Accordingly, a concentration of glucose may be determined that is able to suppress the number of colonies observed on plates with yeast expressing the interacting partners, while maintaining the number of colonies observed on plates expressing only one of the partners. This will determine the optimum gene expression level to select for protein interactions that does not result in auto-activation of the reporter gene.

Using various concentrations of glucose, galactose and FOA, we were able to determine that the optimal conditions for a reverse two-hybrid screen to identify antagonists of the interaction between SCL and E47 was 0.04% FOA, 0.06% galactose and 0.05% glucose.

### Example 2

### Determining the Concentration of Uracil that Reduces Background while Maintaining Plating Efficiency in a Reverse Two-Hybrid Screen

A reverse two-hybrid assay was performed using the interacting proteins JUN1 (amino acids 187-334, inclusive, of the JUN protein) and JUNZ (amino acids 259-309, inclusive, of the JUN protein) both of which comprise the leucine zipper domain of c-Jun (ie the region necessary for self dimerization). As a positive control FOSZ (amino acids 111-195, inclusive, of the FOS protein), a known blocker of c-Jun self dimerization was used. As a negative control, the expression vector alone was used. Gene constructs were transformed into cells comprising the negative selectable markers URA3 and CYH2.

Cells comprising only the interacting partners or the interacting partners and FOSZ (19F) or the interacting partners and the negative control (19) were grown in various concentrations of uracil (from 10 mg/L to 0 mg/L). All cells were also plated in the presence of 0.02% galactose; 0.04% FOA; and 5 μg/ml cycloheximide.

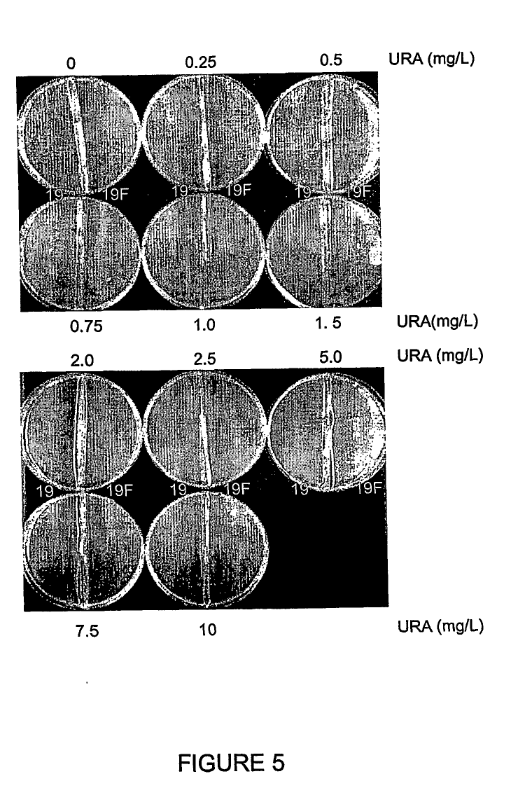

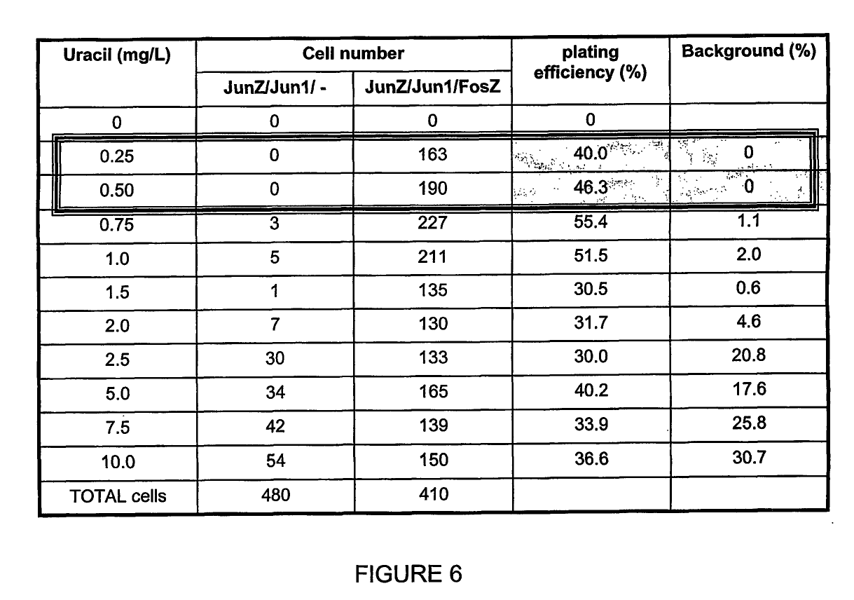

As shown in FIGS. 5 and 6 increased concentrations of uracil increased background and reduced plating efficiency. Background was determined by estimating the number of cells (colonies) that expressed only JUN1 and JUNZ and were still capable of growing in the presence of 5-FOA. Plating efficiency was determined by estimating the number of cells out of the 500 plated that expressed JUN1, JUNZ and FOSZ that were capable of growing in each set of conditions.

As shown in FIG. 6, 0% background and average plating efficiency was attained for concentrations of 0.25 and 0.5 mg/L of uracil.

### Example 3

### Modulating Variables for Reduced Background and Enhanced Plating Efficiency when Using Larger Numbers of Cells

Using the same expression constructs and cells as in Example 2 cells were plated in the presence of increasing concentrations of 5-FOA and uracil. Cells were plated at a density of 1000 or 10000 cells per plate.

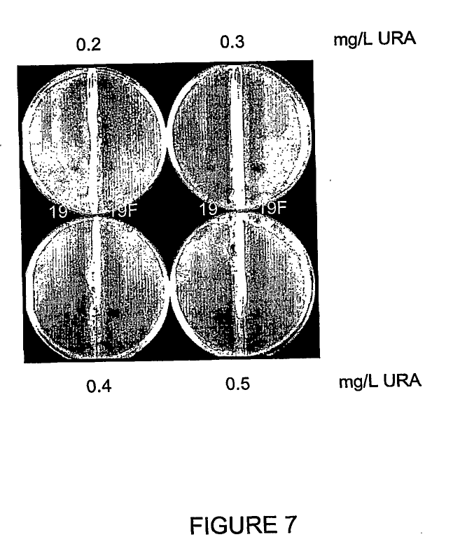

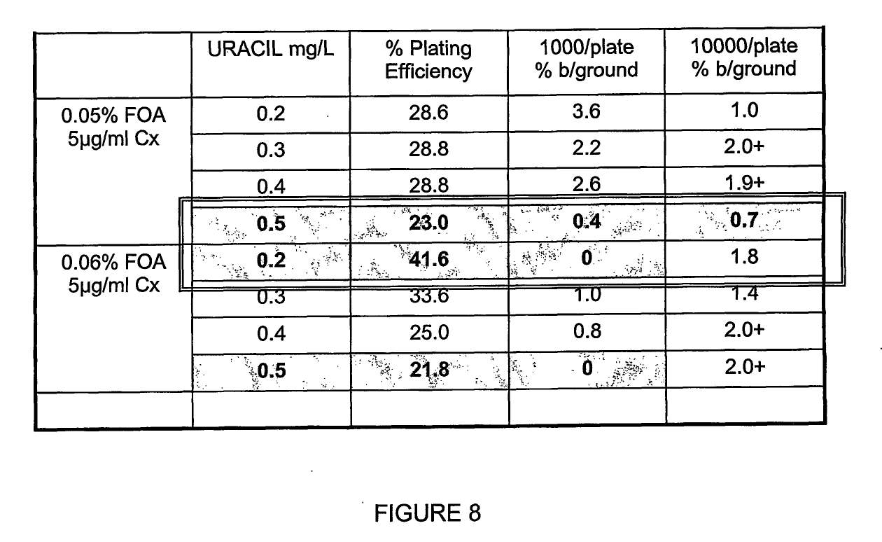

As shown in FIGS. 7 and 8 at a concentration of 0.05% 5-FOA enhanced plating efficiency and reduced background was attained with higher concentrations of uracil, while at 0.06% 5-FOA enhanced plating efficiency and reduced background was attained with lower concentrations of uracil. Both of these conditions were also effective at reducing background and enhancing plating efficiency when larger numbers of cells were screened.

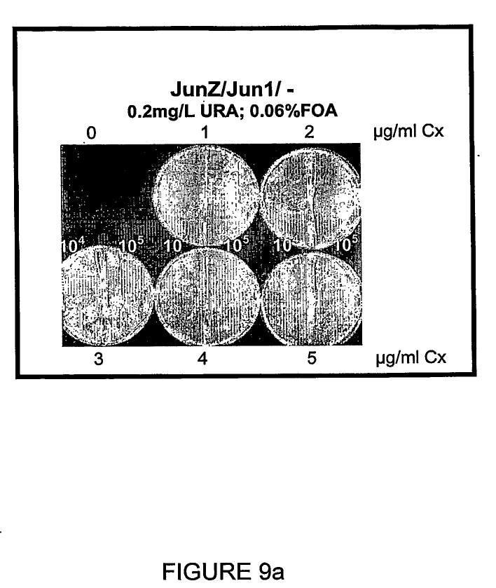

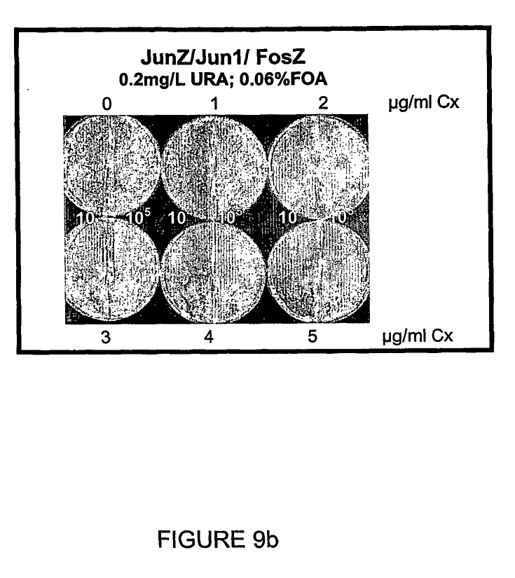

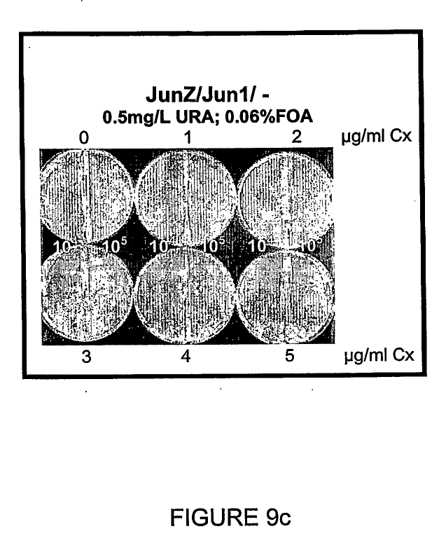

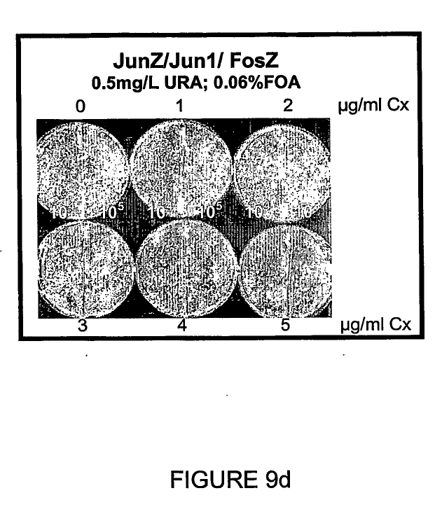

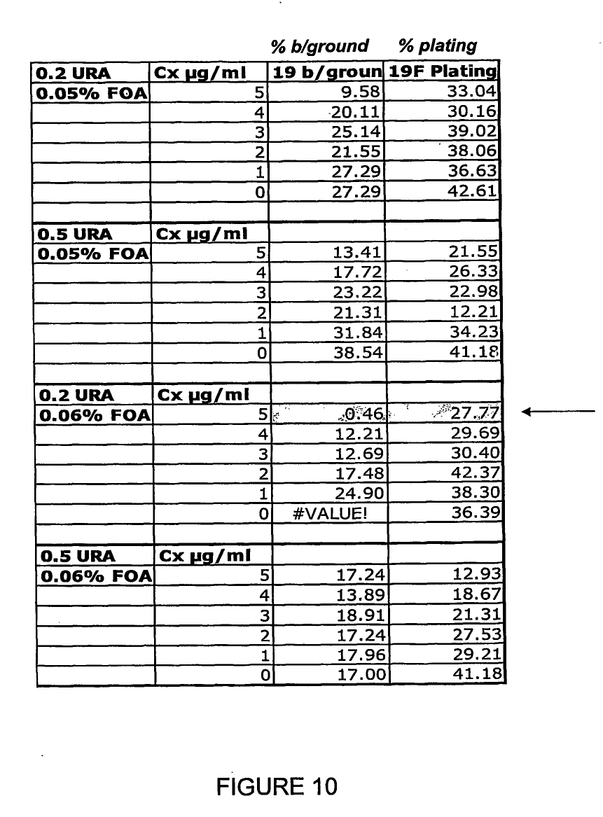

Using these results, a further set of experiments were conducted to determine the effect of modulating the concentration of cycloheximide in the media. Results of these experiments are shown in FIGS. 9 and 10. Using these conditions it was found that increasing the concentration of cycloheximide in media resulted in lower levels of background. Using higher concentrations of cycloheximide (5 μg/ml) and 5-FOA (0.06%) it was found that background levels were reduced to almost negligible levels, while plating efficiency was maintained at a acceptable level (FIG. 10).

### Example 4

### Identification of the Concentration of Phosphate Required to Reduce the Background in a Reverse Two-Hybrid Screen

In order to suppress the expression of the URA 3 reporter molecule the URA3 gene is placed under the control of a modified minimal PHO5 promoter. The TATA box sequence is removed from the PHO5 promoter and replaced with 2 LexA elements. In this way the PHO5 promoter is now inducible through the binding of a LexA DNA binding domain in conjunction with an appropriate activation domain, and as such is useful in a reverse two-hybrid screen.

As the PHO5 promoter is also suppressed by the addition of phosphate salts to the growth media of a cell containing said promoter, it is possible to reduce any background expression of the URA3 reporter molecule. Accordingly, it is necessary to determine the concentration of phosphate salts required to inhibit background expression of the URA3 reporter molecule.

The vectors used in Example 1 are transformed into a yeast strain carrying the mutant PHO5 promoter. Again cells are grown on HWU-complete synthetic media supplemented with various concentrations of sodium orthophosphate.

In this way it is possible to determine if the promoter is able to suppress the activity of the mutant PHO5 promoter in the presence of interacting proteins that bind the LexA operator sequences, in addition to determining if the non-interacting control is able to auto-activate the mutant PHO5 promoter.

The yeast strains are then grown on complete synthetic media supplemented with 5′FOA and various concentrations of glucose and galactose. In this way it is possible to determine the concentration of sodium orthophosphate that is able to reduce the background in a reverse two-hybrid screen.

1. An improved reverse N-hybrid assay for identifying a peptide inhibitor of a DNA-protein or protein-protein interaction in a cell comprising:
(a) determining an amount of a substrate or modulator of a reporter gene required to enhance cell death in the absence of a peptide inhibitor of the DNA-protein or protein-protein interaction; and/or
(b) determining an amount of a substrate or modulator of a reporter gene required to enhance cell survival in the presence of a peptide inhibitor of the DNA-protein or protein-protein interaction; and
(c) performing a reverse N-hybrid assay using an amount of the substrate or modulator as determined at (a) and (b),
wherein expression of the reporter gene modulates cell survival by virtue of encoding a polypeptide that reduces cell growth or viability by providing a target for a cytostatic or cytotoxic compound or by converting the substrate to a cytostatic or cytotoxic product.

2. The improved reverse N-hybrid assay of claim 1 wherein the peptide inhibitor of a DNA-protein or protein-protein interaction is a heterologous peptide that is not endogenous to the cell or capable of modulating expression of the reporter gene.

3. The improved reverse N-hybrid assay of claim 1 wherein the cell is a yeast cell.

4. The improved reverse N-hybrid assay according to claim 1 wherein the background number of cells is determined using a method comprising
(a) culturing a known number of cells that expresses at least one interacting protein of a protein-DNA interaction or at least two proteins of a protein-protein interaction in the presence of a substrate of a reporter gene under conditions sufficient to produce cell death by virtue of reporter gene expression; and
(b) determining the number of cells that survive and/or grow wherein any cells that survive and/or grow in the presence of the substrate of the reporter gene are background.

5. The improved reverse N-hybrid assay according to claim 1 wherein the plating efficiency is determined by a method comprising:
(a) culturing a known number of cells that express at least one interacting protein of a protein-DNA interaction and a peptide inhibitor of the protein-DNA interaction or at least two proteins of a protein-protein interaction and a peptide inhibitor of the protein interaction in the presence of a substrate of a reporter gene under conditions sufficient to produce cell death by virtue of reporter gene expression; and
(b) determining the number of cells that survive and/or grow compared to the number of cells cultured wherein a decrease in the number of cells that survive and/or grow compared to the number of cells cultured indicates a reduction in plating efficiency.

6. The improved reverse N-hybrid assay of claim 4 wherein conditions sufficient to produce cell death by virtue of reporter gene expression comprise incubating the cells in an amount of a toxigenic substrate of the reporter gene under conditions sufficient for the reporter gene to be capable of being expressed in the cell.

7. The improved reverse N-hybrid assay of claim 6 wherein the cell is a yeast cell and the toxigenic substrate is 5-fluororotic acid (5-FOA) and the reporter gene is URA3.

8. The improved reverse N-hybrid assay of claim 6 wherein the cell is a yeast cell and the toxigenic substrate is cycloheximide and the reporter gene is CYH2.

9. The improved reverse N-hybrid assay of claim 6 wherein the cell is a yeast cell and the toxigenic substrate is α-aminoapidate and the reporter gene is LYS2.

10. The improved reverse N-hybrid assay of claim 6 wherein conditions sufficient to produce cell death by virtue of reporter gene expression comprise incubating the cells in an amount of a plurality of toxigenic substrates of a plurality of reporter genes under conditions sufficient for each reporter gene to be capable of being expressed in the cell.

11. The improved reverse N-hybrid assay of claim 10 wherein the cell is a yeast cell and the plurality of reporter genes is selected from the group consisting of URA3, CYH2, LYS2 and wherein the plurality of toxigenic substrates is selected from the group consisting of 5-fluororotic acid, cycloheximide and α-aminoapidate.

12. The improved reverse N-hybrid assay of claim 4 wherein conditions sufficient to produce cell death by virtue of reporter gene expression comprise incubating the cells in media lacking an amount of a compound required for cell survival and complemented by a product of reporter gene expression under conditions sufficient for the reporter gene to be capable of being expressed in the cell.

13. The improved reverse N-hybrid assay of claim 12 wherein the cell is a yeast cell and the compound required for cell survival is uracil and the reporter gene is URA3.

14. The improved reverse N-hybrid assay of claim 4 wherein conditions sufficient to produce cell death by virtue of reporter gene expression comprise incubating the cells in an amount of a compound sufficient to modulate expression of a gene selected from the group consisting of a reporter gene and a gene encoding a binding partner to the DNA-protein or protein-protein interaction.

15. The improved reverse N-hybrid assay of claim 14 wherein the gene encoding a binding partner comprises a promoter that is regulated by the compound thereby modulating expression of the reporter gene by virtue of modulating the amount of a binding partner that regulates reporter gene expression in the cell.

16. The improved reverse N-hybrid assay of claim 14 wherein the reporter gene comprises a promoter that is regulated by the compound thereby modulating expression of the reporter gene in the cell.

17. The improved reverse N-hybrid assay of claim 15 wherein the gene encoding a binding partner or the reporter gene is placed operably under the control of a promoter that is induced in the presence of the compound.

18. The improved reverse N-hybrid assay of claim 17 wherein the compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

19. The improved reverse N-hybrid assay of claim 15 wherein the gene encoding a binding partner or the reporter gene is placed operably under the control of a promoter that is repressed in the presence of the compound.

20. The improved reverse N-hybrid assay of claim 19 wherein the compound is glucose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

21. The improved reverse N-hybrid assay of claim 19 wherein the compound is phosphate and the promoter is a PHO5 promoter (SEQ ID NO: 6).

22. The improved reverse N-hybrid assay of claim 4 wherein conditions sufficient to produce cell death by virtue of reporter gene expression comprise incubating the cells in an amount of a plurality of compounds sufficient to modulate expression of a gene selected from the group consisting of a reporter gene and a gene encoding a binding partner to the DNA-protein or protein-protein interaction.

23. The improved reverse N-hybrid assay of claim 22 wherein the gene encoding a binding partner comprises a promoter that is regulated by the plurality of compounds thereby modulating expression of the reporter gene by virtue of modulating the amount of a binding partner that regulates reporter gene expression in the cell.

24. The improved reverse N-hybrid assay of claim 22 wherein the reporter gene comprises a promoter that is regulated by the plurality of compounds thereby modulating expression of the reporter gene in the cell.

25. The improved reverse N-hybrid assay of claim 22 wherein the gene encoding a binding partner or the reporter gene comprises a promoter that is induced in the presence of a compound and repressed in the presence another compound of said plurality of compounds.

26. The improved reverse N-hybrid assay of claim 25 wherein a compound is glucose and another compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

27. The improved reverse N-hybrid assay of claim 14 wherein the amount of one or more compounds sufficient to modulate expression of a binding partner to a DNA-protein or a protein-protein interaction is determined by a method comprising:
(a) culturing a cell that does not express a protein interacting partner of a DNA-protein interaction or expresses one protein interacting partner of a protein-protein interaction in the presence of the one or more compounds that modulates expression of the protein binding partner and in the absence of a peptide inhibitor of the DNA-protein or protein-protein interaction and determining the amount of the compound that reduces cell death under conditions sufficient to produce cell death by virtue of reporter gene expression; and
(b) culturing a cell that expresses a protein interacting partner of a DNA-protein interaction or expresses the proteins of a protein-protein interaction in the presence of the compound that modulates expression of the protein binding partner and in the absence of a peptide inhibitor of the DNA-protein or protein-protein interaction and determining the amount of the compound that enhances cell death under conditions sufficient to produce cell death by virtue of reporter gene expression,
(c) determining an amount of the substrate that reduces cell death at (a) and enhances cell death at (b).

28. An improved reverse N-hybrid assay for identifying a peptide inhibitor of a DNA-protein or protein-protein interaction in a yeast cell comprising:
(a) determining an amount of uracil required to enhance cell death in a cell in the absence of a peptide inhibitor of the DNA-protein or protein-protein interaction wherein the cell is cultured under conditions sufficient to produce cell death; and
(b) determining an amount of uracil required to enhance cell survival in the presence of a peptide inhibitor of the DNA-protein or protein-protein interaction wherein the cell is cultured under conditions sufficient to produce cell death; and
(c) performing a reverse N-hybrid assay using an amount of uracil as determined at (a) and (b),

29. An improved reverse N-hybrid assay for identifying a peptide inhibitor of a DNA-protein or protein-protein interaction in a yeast cell comprising:
(a) determining an amount of glucose and galactose required to modulate URA3 reporter gene expression and enhance cell death in the absence of a peptide inhibitor of the DNA-protein or protein-protein interaction;
(b) determining an amount of glucose and galactose required to modulate URA3 reporter gene expression and enhance cell survival in the presence of a peptide inhibitor of the DNA-protein or protein-protein interaction; and
(c) performing a reverse N-hybrid assay using an amount of the substrate or modulator as determined at (a) and (b),
wherein expression of the URA3 reporter gene modulates cell survival by virtue of encoding a polypeptide that reduces cell growth or viability by converting 5-fluororotic acid to a cytostatic or cytotoxic product.

30. An improved reverse N-hybrid assay for identifying a peptide inhibitor of a DNA-protein or protein-protein interaction in a yeast cell comprising:
(a) determining an amount of uracil and an amount of 5-fluororotic acid required to enhance cell death in a cell in the absence of a peptide inhibitor of the DNA-protein or protein-protein interaction wherein the cell is cultured under conditions sufficient to produce cell death; and
(b) determining an amount of uracil and an amount of 5-fluororotic acid required to enhance cell survival in the presence of a peptide inhibitor of the DNA-protein or protein-protein interaction wherein the cell is cultured under conditions sufficient to produce cell death; and
(c) performing a reverse N-hybrid assay using an amount of uracil and an amount of 5-fluororotic acid as determined at (a) and (b).

31. An improved reverse N-hybrid assay for identifying a peptide inhibitor of a DNA-protein or protein-protein interaction in a yeast cell comprising:
(a) determining an amount of uracil and an amount of 5-fluororotic acid and an amount of cycloheximide required to enhance cell death in a cell in the absence of a peptide inhibitor of the DNA-protein or protein-protein interaction wherein the cell is cultured under conditions sufficient to produce cell death; and
(b) determining an amount of uracil and an amount of 5-fluororotic acid and an amount of cycloheximide required to enhance cell survival in the presence of a peptide inhibitor of the DNA-protein or protein-protein interaction wherein the cell is cultured under conditions sufficient to produce cell death; and
(c) performing a reverse N-hybrid assay using an amount of uracil and an amount of 5-fluororotic acid as determined at (a) and (b).

32. An improved forward N-hybrid assay for identifying a heterologous peptide or protein capable of binding to the DNA or protein in a cell comprising:
(a) determining an amount of a modulator of a reporter gene required to inhibit cell growth and/or survival in the absence of a heterologous peptide or protein capable of binding to the DNA or protein in a cell;
(b) determining an amount of a modulator of a reporter gene required to enhance cell growth and/or survival in the presence of a heterologous peptide or protein capable of binding to the DNA or protein in a cell; and
(c) performing a forward N-hybrid assay using an amount of the modulator as determined at (a) and (b);
wherein expression of the reporter gene modulates cell survival by virtue of encoding a polypeptide required for cell growth and/or survival.

33. The improved forward N-hybrid assay of claim 32 wherein the cell is a yeast cell.

34. The improved forward N-hybrid assay according to claim 32 wherein the background number of cells is determined using a method comprising:
(a) culturing a known number of cells that do not express a protein binding partner of a protein-DNA interaction or expresses an interacting protein of a protein-protein interaction in the presence of a substrate of a reporter gene under conditions sufficient to produce cell growth and/or survival by virtue of reporter gene expression; and
(b) determining the number of cells that survive and/or grow wherein any cells that survive and/or grow in the presence of the substrate of the reporter gene are background.

35. The improved forward N-hybrid assay according to claim 32 wherein the plating efficiency is determined by a method comprising:
(a) culturing a known number of cells that express a protein binding partner of a protein-DNA interaction or express the proteins of a protein-protein interaction in the presence of a substrate of a reporter gene, under conditions sufficient to produce cell growth and/or survival by virtue of reporter gene expression; and
(b) determining the number of cells that survive and/or grow compared to the number of cells cultured wherein a decrease in the number of cells that survive and/or grow compared to the number of cells cultured indicates a reduction in plating efficiency.

36. The improved forward N-hybrid assay of claim 34 wherein conditions sufficient to produce cell growth and/or survival by virtue of reporter gene expression comprise incubating the cells in an amount of a compound sufficient to modulate expression of a gene selected from the group consisting of a reporter gene and a gene encoding a binding partner to the DNA-protein or protein-protein interaction.

37. The improved forward N-hybrid assay of claim 36 wherein the gene encoding a binding partner comprises a promoter that is regulated by the compound thereby modulating expression of the reporter gene by virtue of modulating the amount of a binding partner that regulates reporter gene expression in the cell.

38. The improved forward N-hybrid assay of claim 36 wherein the reporter gene comprises a promoter that is regulated by the compound thereby modulating expression of the reporter gene in the cell.

39. The improved forward N-hybrid assay of claim 37 wherein the gene encoding a binding partner or the reporter gene comprises a promoter that is induced in the presence of the compound.

40. The improved forward N-hybrid assay of claim 39 wherein the compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

41. The improved forward N-hybrid assay of claim 37 wherein the gene encoding a binding partner or the reporter gene comprises a promoter that is repressed in the presence of the compound.

42. The improved forward N-hybrid assay of claim 41 wherein the compound is glucose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

43. The improved forward N-hybrid assay of claim 41 wherein the compound is phosphate and the promoter is a PHO5 promoter (SEQ ID NO: 6).

44. The improved forward N-hybrid assay of claim 34 wherein conditions sufficient to produce cell growth and/or survival by virtue of reporter gene expression comprise incubating the cells in an amount of a plurality of compounds sufficient to modulate expression of a gene selected from the group consisting of a reporter gene and a gene encoding a binding partner to the DNA-protein or protein-protein interaction.

45. The improved forward N-hybrid assay of claim 44 wherein the gene encoding a binding partner comprises a promoter that is regulated by the plurality of compounds thereby modulating expression of the reporter gene by virtue of modulating the amount of a binding partner that regulates reporter gene expression in the cell.

46. The improved forward N-hybrid assay of claim 44 wherein the reporter gene comprises a promoter that is regulated by the plurality of compounds thereby modulating expression of the reporter gene in the cell.

47. The improved forward N-hybrid assay of claim 44 wherein the gene encoding a binding partner or the reporter gene comprises a promoter that is induced in the presence of a compound and repressed in the presence another compound of said plurality of compounds.

48. The improved forward N-hybrid assay of claim 47 wherein a compound is glucose and another compound is galactose and the promoter is selected from the group consisting of a GAL1/GAL10 promoter (SEQ ID NO: 1), a GAL2 promoter (SEQ ID NO: 2), a GAL4 promoter (SEQ ID NO: 3), a GAL7 promoter (SEQ ID NO: 4) and a MEL1 promoter (SEQ ID NO: 5).

49. The improved reverse N-hybrid assay of claim 37 wherein the amount of one or more compounds sufficient to modulate expression of a binding partner to a DNA-protein or a protein-protein interaction is determined by a method comprising:
(a) culturing a cell that does not express a protein binding partner of a DNA-protein interaction or expresses a protein binding partner of a protein-protein interaction in the presence of the one or more compounds that modulate expression of the protein binding partner and determining the amount of the compound that suppresses cell growth and/or survival under conditions sufficient to produce cell growth and/or survival by virtue of reporter gene expression; and
(b) culturing a cell that expresses a protein binding partner of a DNA-protein interaction or expresses the proteins of a protein-protein interaction in the presence of the one or more compounds that modulate expression of the protein binding partner and determining the amount of the one or more compounds sufficient to produce cell growth and/or survival by virtue of reporter gene expression,
wherein an amount of the substrate that suppresses cell growth and/or survival at (a) and produces cell growth and/or survival at (b) is an amount of the compound that enhances expression of the protein binding partner thereby enhancing cell growth and/or survival in the presence of a DNA-protein or protein-protein interaction and does not compromise or inhibit cell survival in the absence of DNA-protein or protein-protein interaction.

50. The improved reverse N-hybrid assay according to claim 1 wherein the protein binding partners are expressed operably under control of independently regulatable promoters.

51. The improved reverse N-hybrid assay or the improved forward N-hybrid assay according to claim 50 wherein a promoters are different promoters.
